(19) United States
(12) Patent Application Publication
(10) Pub. No.: US 2024/0404628 A1
(43) Pub. Date: Dec. 5, 2024

(54) METHODS TO DETERMINE MATERNITY, PATERNITY, OR PARENTAGE AND COMPUTER SYSTEMS FOR IMPLEMENTATION THEREOF

(71) Applicant: The Chinese University of Hong Kong, Hong Kong (CN)

(72) Inventors: Zirui DONG, Hong Kong (CN); Kwong Wai CHOY, Hong Kong (CN); Keying LI, Hong Kong (CN); Matthew Hoi Kin CHAU, Hong Kong (CN)

(21) Appl. No.: 18/672,159

(22) Filed: May 23, 2024

Related U.S. Application Data
(60) Provisional application No. 63/504,845, filed on May 30, 2023.

(51) Int. Cl. G16B 20/20; G16B 30/10

(52) U.S. Cl. CPC G16B 20/20; G16B 30/10

(57) ABSTRACT

The subject invention pertains to the field of healthcare informalities and provides information and communication (ICT) technology useful for methods and tools for paternity and/or maternity testing. An analytical pipeline based on low-pass GS (e.g., 1-fold read depth), referred to herein as LpPat, is provided for paternity and/or maternity testing (e.g., with trio-based and duo-based analytical modes). By down-sampling the read-depth from 10 trios with confirmed paternity and maternity, an optimal read depth of 1-fold is demonstrated across other sequencing parameters with a turnaround time for the analysis of less than one hour. The robust performance has been validated by another 170 trios sequenced from different library construction methods and platforms. The algorithmic analysis provides a rapid, cost effective, and sequencing-platform-neutral paternity and/or maternity test, based on low-pass genetic sequencing.

### CROSS-REFERENCE TO RELATED APPLICATION

This application claims the benefit of U.S. Provisional Application Ser. No. 63/504,845, filed May 30, 2023, which is hereby incorporated by reference in its entirety including any tables, figures, or drawings.

### TECHNICAL FIELD OF THE INVENTION

The subject invention relates generally to healthcare informatics, i.e., information and communication technology (ICT) specially adapted for the handling or processing of medical or healthcare data. More specifically, the subject invention relates to ICT specially adapted for medical identification or diagnosis, medical simulation, or medical data mining.

### BACKGROUND OF THE INVENTION

With recent rapid advancements in sequencing technologies, genome sequencing (GS) has been widely used in a clinical setting (Prokop, et al., 2018), to provide a higher diagnostic yield of genetic abnormalities compared with traditional tests among high-risk pregnancies (Cao, et al., 2022; Choy, et al., 2019; Zhou, et al., 2021). In particular, a trio-based (proband and biological parents) GS testing determines the mode of inheritance of genomic variants, assisting variant classification and the interpretation of clinical significance (Zhou, et al., 2021). However, submission of one or both non-biological parents would cause misattributed parentage (MP), possibly resulting in misdiagnosis. Based on the estimation from the American Society of Human Genetics, misattributed paternity occurs at a rate between 1% and 10% (Prero, et al., 2019). It is understood that the rate of MP might be increased alongside the increasing rate of adoption or gamete donation due to infertility. As trio-based high read-depth GS can be costly, there is a need for a rapid and cost-effective paternity/maternity test as a quality control step, in particular to avoid sample mix-up.

Currently, a polymerase chain reaction (PCR) based method utilizing short tandem repeats (STRs) serves as the gold-standard method for paternity testing (Ou and Qu, 2020). However, challenges remain. For instance, stutter artefacts generated during amplification due to repetitive motifs, and mutations in STRs could interfere the probability of paternity calculation. In comparison, although single nucleotide polymorphism (SNP) typing has been recently adopted for forensic science by genotyping of a list/panel of SNPs (Schwark, et al., 2012), allele frequencies among different races have not been evaluated with the existing panels (Chandra, et al., 2022; Tam, et al., 2020). Although these methods serve as the gold-standard for paternity/maternity testing, some laboratories might not have the capacity and/or willingness to perform such labor-intensive and time-consuming experiments such as GS or exome sequencing (ES). In contrast, microhaplotype, which requires at least two SNPs within 200 bp, has been introduced. However, it also relies on genotyping approaches such as high read-depth sequencing (GS/ES) (Shen, et al., 2021). Therefore, there is a clear need for a rapid, accurate and cost-effective paternity/maternity test based on GS.

Low-pass GS, characterized by shallow coverage high throughput sequencing (0.1-4-fold read-depth) has demonstrated its capability and feasibility in the detection of copy number variants (Chaubey, et al., 2020; Dong, et al., 2016; Liang, et al., 2014; Wang, et al., 2020), structural rearrangements (Dong, et al., 2018; Redin, et al., 2017) and regions with absence of heterozygosity (Chaubey, et al., 2020; Dong, et al., 2021). However, unlike targeting sequencing of panels with pre-selected markers, the detection capability of targeted single-nucleotide variants (SNVs) by low-pass genome sequencing is limited. Low-pass GS relies on shotgun or random sequencing across nearly the entire genome, resulting in relatively even coverage across the genome. The variation in sequencing coverage between samples and batches can make it challenging to obtain adequate reads for determining genotypes at predetermined sites (FIG. 1A). In addition, genotyping using low-coverage GS data is error prone due to the lack of coverage. Heterozygous SNVs could be mistakenly detected by low-pass GS as homozygous SNVs due to the insufficient reads supporting the alternate allele.

Similarly, SNVs could be missed if the coverage of the mutant allele is insufficient (FIG. 1B). Lastly, predetermining regions with high coverage across different samples and batches could be problematic because they could represent biases caused by systematic errors during alignment.

### BRIEF SUMMARY OF THE INVENTION

Embodiments of the subject invention provide an analytical pipeline (LpPat) for a rapid, cost-effective, and sequencing platform neutral paternity test based on low-pass GS, which is about 1-fold read-depth to about 15-fold read-depth. In certain embodiments, single-end sequence reads or paired-end sequence reads of polynucleotides can be used. In certain embodiments, the sequence read length can be at least 1 2, 3, 4, 5, 6, 7, 8, 9, 10, about 15, about 25, about 50, about 75, about 100, about 125, about 150, about 175, about 200, about 225, about 250, about 500, about 1000, or longer bases or base pairs. In certain embodiments, the number of reads is variable (because the read-lengths are variable). For example, the number of reads or read pairs is at least about 100,000, about 250,000, about 500,000, about 750,000, about 1 million, about 2.5 million, about 5 million, about 7.5 million, about 10 million, about 15 million, about 20 million, about 25 million, about 30 million, about 40 million, about 50 million, or more. For another example, the steps of the subject invention comprise about 30 million reads of 100 bases obtained by single-end sequencing, which is equal to 1-fold read depth: (100 bases*30 millions)/3G human genome size). For yet another example, the steps of the subject invention comprise about 10 million read-pairs of 150 base pairs obtained using paired-end sequencing: (150 bp*10 million*2)/3G, which is also 1-fold read-depth. Embodiments can provide analysis in two scenarios: a duo analysis mode designed for the submission of a pair of samples (proband and a presumed parent), and a trio analysis mode designed for the submission of three samples (proband and two presumed parents).

Embodiments of the subject invention provide a method of detecting parental inheritance of genotypes for paternity testing in biological samples from subjects, comprising:
(i) aligning sequence reads from low-pass genome sequencing of genomic DNA of biological samples to a human genome reference, and selecting and sorting sequence reads aligned to the human genome reference based on the aligned chromosome and genomic coordinates;
(ii) identifying single-nucleotide variants (SNVs) in the aligned sequence reads, wherein a single-nucleotide variant at each site has a mutant base type different from the base type at the corresponding site from the human genome reference;
(iii) identifying homozygous SNVs and diploid heterozygous SNVs from the SNVs identified in step (iii), wherein:
   a) a homozygous SNV is defined based on the percentage of sequence reads supporting the mutant base type that is different from the base type at the corresponding site from the human genome reference being 100%, and
   b) a diploid heterozygous SNV is defined based on the percentage of sequence reads supporting the mutant base type that is different from the base type at the corresponding site from the human genome reference being no less than 25% and no larger than 75%;
(iv) determining an inconsistent rate of base-type inheritance from the SNVs identified in step (iii) for the two analytical models, wherein
   for trio-based analysis, (a) the number of loci that both parents were in homozygous manner but with different genotypes in the i-^{th} chromosome was denoted as Aⁱ; (b) among them, the number of SNVs that were homozygous in the proband but with different genotypes from the presumed father was denoted as pi (for paternity test) or mi (for maternity test); (c) the inconsistent rate of paternal inheritance αi in chromosome i was calculated by formula (1):

$$α i = \frac{pi}{Ai} \qquad ( 1 )$$

(d) The paternity was determined by formula (2) using the average rate λ̅pat across all autosomal chromosomes:

$$\overline{λ} pat = \frac{{\sum }_{i = 1}^{n = 22} α i}{22} \qquad ( 2 )$$

The same process is applied for maternity test. The inconsistent rate of maternal inheritance βi in chromosome i was calculated by formula (3), while the maternity was determined by formula (4) using the average rate λ̅mat across all autosomal chromosomes:

$$β i = \frac{mi}{Ai} \qquad ( 3 )$$

$$\overline{λ} mat = \frac{{\sum }_{i = 1}^{n = 22} β i}{22} \qquad ( 4 )$$

For duo-based analysis, (a) the number of homozygous SNVs in both proband and the presumed parent in chromosome i was denoted as Adi; (b) among them, the number of homozygous SNVs that were with different genotypes between the proband and the presumed father was denoted as qi; (c) the inconsistent rate γi of paternal inheritance in chromosome i was calculated by formula (5):

$$γ i = \frac{qi}{Adi} \qquad ( 5 )$$

(d) The paternity was determined by formula (6) using the average rate λ̅pat across all autosomal chromosomes:

$$\overline{λ} pat = \frac{{\sum }_{i = 1}^{n = 22} γ i}{22} \qquad ( 6 )$$

Moreover, maternity was determined by the same method for the paternity determination.

In another embodiment, a computer system is provided for calculating the inconsistent rate of base-type inheritance for paternity testing in biological samples from subjects, comprising a processor and a memory storing a plurality of instructions, wherein the processor, upon processing the instructions, is configured to perform the following steps:
(i) aligning sequence reads from low-pass genome of genomic DNA of biological samples to a human genome reference, and selecting and sorting sequence reads aligned to the human genome reference based on the aligned chromosome and genomic coordinates;
(ii) identifying single-nucleotide variants (SNVs) in the aligned sequence reads, wherein a single-nucleotide variant at each site has a mutant base type that is different from the base type at the corresponding site from the human genome reference;
(iii) identifying homozygous SNVs and/or diploid heterozygous SNVs from the SNVs identified in step (ii), wherein a homozygous SNV is defined based on the percentage of sequence reads supporting the mutant base type that is different from the base type at the corresponding site from the human genome reference being 100%, a diploid heterozygous SNV is defined based on the percentage of sequence reads supporting the mutant base type that is different from the base type at the corresponding site from the human genome reference being no less than 25% and no larger than 75%;
(iv) determining an inconsistent rate of base-type inheritance from the SNVs identified in step (iii) for the two analytical models, wherein for the trio-based analysis, (a) the number of loci that both parents were in homozygous manner but with different genotypes in the i-^{th} chromosome was denoted as Aⁱ; (b) among them, the number of SNVs that were homozygous in the proband but with different genotypes from the presumed father was denoted as pi (mi: for maternity test); (c) the inconsistent rate of paternal inheritance αi in chromosome i was calculated by the formula (1); (d) the paternity was determined by the formula (2) using the average rate λ̅pat across all autosomal chromosomes; the same process can be applied to maternity test. The inconsistent rate of maternal inheritance βi in chromosome i was calculated by the formula (3), while the maternity was determined by the formula (4) using the average rate λ̅mat across all autosomal chromosomes; and for the duo-based analysis, (a) the number of homozygous SNVs in both proband and the presumed parent in chromosome i is denoted as Adi; (b) among them, the number of homozygous SNVs that were with different genotypes between the proband and the presumed father was denoted as qi; (c) the inconsistent rate γi of paternal inheritance in chromosome i was calculated by the formula (5); (d) the paternity was determined by the formula (6) using the average rate λ̅pat across all autosomal chromosomes; the maternity was determined by the same method for the paternity determination.

In a third embodiment, a computer readable medium storing a plurality of instructions is provided, wherein the plurality of instructions, when executed by one or more processors, perform an operation including the following steps:
(i) aligning sequence reads from low-pass genome sequencing of genomic DNA of biological samples to a human genome reference, and selecting and sorting sequence reads aligned to the human genome reference based on the aligned chromosome and genomic coordinates;
(ii) identifying single-nucleotide variants (SNVs) in the aligned sequence reads, wherein a single-nucleotide variant at each site has a mutant base type different from the base type at the corresponding site from the human genome reference;
(iii) identifying homozygous SNVs and/or diploid heterozygous SNVs from the SNVs identified in step (ii), wherein a homozygous SNV is defined based on the percentage of sequence reads supporting the mutant base type that is different from the base type at the corresponding site from the human genome reference being 100%, a diploid heterozygous SNV is defined based on the percentage of sequence reads supporting the mutant base type that is different from the base type at the corresponding site from the human genome reference being no less than 25% and no larger than 75%;
(iv) determining an inconsistent rate of base-type inheritance from the SNVs identified in step (iii) for the two analytical models, wherein for the trio-based analysis, (a) the number of loci that both parents were in homozygous manner but with different genotypes in the i-^{th} chromosome was denoted as Aⁱ; (b) among them, the number of SNVs that were homozygous in the proband but with different genotypes from the presumed father was denoted as pi (mi: for maternity test); (c) the inconsistent rate of paternal inheritance αi in chromosome i was calculated by the formula (1); (d) the paternity was determined by the formula (2) using the average rate λ̅pat across all autosomal chromosomes; the same process can be applied for maternity test, the inconsistent rate of maternal inheritance βi in chromosome i was calculated by the formula (3), while the maternity was determined by the formula (4) using the average rate λ̅mat across all autosomal chromosomes; for the duo-based analysis, (a) the number of homozygous SNVs in both proband and the presumed parent in chromosome i is denoted as Adi; (b) among them, the number of homozygous SNVs that were with different genotypes between the proband and the presumed father was denoted as qi; (c) the inconsistent rate γi of paternal inheritance in chromosome i was calculated by the formula (5); (d) the paternity was determined by the formula (6) using the average rate λ̅pat across all autosomal chromosomes; the maternity was determined by the same method applied for the paternity determination.

### BRIEF DESCRIPTION OF THE DRAWINGS

FIG. 1A illustrates certain difficulties of SNV detection by low-pass genome sequencing, showing a comparison of detecting pre-selected sites by high read-depth GS and low-pass GS. For the analysis with high read-depth GS, genotyping is precise to indicate heterozygous or homozygous SNVs due to the adequate read-depth. Whilst by low read-depth GS, due to the randomly distributed reads, SNVs might not be detected (indicated by question mark), misassigned as homozygous (indicated by green font), or incorrectly detected (indicated by red font) between different batches.

FIG. 1B illustrates certain possible scenarios of homozygous SNV detection caused by insufficient reads with low-pass GS. For the locus, the genotype in the proband should be heterozygous AT based on the genotypes in the parents. Apparently, an equal number of reads supporting the reference and mutant base types are shown in high read-depth GS. In the scenarios of low-pass GS, the genotype might be misassigned as homozygous AA or TT due to the insufficient reads in supporting the reference or mutant base type.

FIG. 2A shows a diagram of paternity testing by low-pass GS in the trio-based analysis according to an embodiment of the subject invention. In the upper panel, loci that are homozygous in both parents but with different genotypes are indicated by green frames. In the middle panel, those loci that exhibit inconsistency of paternal or maternal inheritance are shown in blue or orange frames.

FIG. 2B shows the inconsistent rate of base-type inheritance from a trio that the biological background is from one of the three scenarios.

FIGS. 2C-2D show a diagram of paternity testing by low-pass GS in duo-based analysis according to an embodiment of the subject invention. In the upper panel, loci that are homozygous in the proband and the presumed father/mother are indicated by green frames. In the middle panel, those loci that exhibit inconsistency of paternal/maternal inheritance are shown in blue frames.

FIG. 2D shows the inconsistent rate of base-type inheritance from a dual setting that the biological background is from one of the two scenarios.

FIG. 3 illustrates the analytical process and turn-around-time estimation for LpPat according to an embodiment of the subject invention. The upper panel indicates the shared procedures between two modes, and the rest of procedures in trio-based and duo-based analysis are shown in the middle or bottom, respectively. Each font in red indicates the average turn-around-time for that procedure.

FIGS. 4A-4D show certain biological relationship among the trios confirmed by QF-PCR. QF-PCR (quantitative fluorescent polymerase chain reaction) with STR (short tandem repeat) marker in family of 22C1246 (proband), 22C1607 (the presumed mother), and 22C1608 (the presumed father) shown in the upper, middle, and bottom panel, respectively, according to an embodiment of the subject invention. Each pair of a locus is linked, and the allele in the proband inherited from the father is indicated by a blue arrow, while the other allele in the proband inherited from the mother is indicated by a red arrow. FIGS. 4B, 4C, and 4D show an expanded or zoomed in view of the areas indicated as Detail 4B, Detail 4C, and Detail 4D, respectively.

FIGS. 5A-5H illustrate confirmation of the biological relationship by comparing the genotypes among the proband and the presumed parents from the 1000 Genomes Project, according to an embodiment of the subject invention. Integrative Genomics Viewer view of genotypes among the proband (upper panel), the presumed mother (middle panel) and the presumed father (lower panel) is shown. FIGS. 5A-5D show that the locus is rs1490413 with location of 4307263 in hg38 and of 4367323 in GRCh37/hg19. FIGS. 5E-5G show that the locus is rs1335873 with location of 20327585 in GRCh38/hg38 and of 20901724 in GRCh37/hg19. FIGS. 5B, 5C, and 5D show an expanded or zoomed in view of the areas indicated as Detail 5B, Detail 5C, and Detail 5D, respectively. FIGS. 5F, 5G, and 5H show an expanded or zoomed in view of the areas indicated as Detail 5F, Detail 5G, and Detail 5H, respectively.

FIGS. 6A-6B illustrate determination of the optimal read-depth required for the analysis, according to an embodiment of the subject invention, including a boxplot of the inconsistent rate of parental inheritance among paired-end 100 bp data with different read-depths with small insert libraries and data with 1-fold read-depth from mate-pair library constructions in the trio-based analysis (FIG. 6A) and in the duo-based analysis (FIG. 6B).

FIGS. 7A-7D show determination of the optimal read-depth required for the analysis, according to an embodiment of the subject invention, including a boxplot of the inconsistent rate of parental inheritance among data with different read-depths with small insert libraries and paired-end 150 bp in the trio-based analysis (FIG. 7A) and in the duo-based analysis (FIG. 7B), or single-end 150 bp in the trio-based analysis (FIG. 7C) and in the duo-based analysis (FIG. 7D).

FIGS. 8A-8F show an evaluation of the performance among 170 trios with data from different library construction methods and sequencing platforms, according to an embodiment of the subject invention, including a boxplot of the inconsistent rate of parental inheritance among 100 trios with small-insert libraries and sequenced by MGISeq-2000, 20 trios with mate-pair libraries and sequenced by MGISeq-2000, and 50 trios with small-insert libraries and sequenced in NovaSeq in the trio-based analysis (FIG. 8A) and in the duo-based analysis (FIG. 8B). The outlier in both analyses is indicated by the red arrow in each FIG. 8C. QF-PCR with STR markers for the validation in family of 22C1246 (proband), 22C1607 (the presumed mother), and 22C1608 (the presumed father) are shown. Two pairs of loci are shown to indicate non-maternity. Each pair of a locus is linked, and the allele in the proband inherited from the father is indicated by a blue arrow, while the other allele in the proband is indicated by a red arrow, which is not presented in the mother. FIGS. 8D, 8E, and 8F show an expanded or zoomed in view of the areas indicated as Detail 8D, Detail 8E, and Detail 8F, respectively.

FIG. 9 shows frequency distribution of the recurrent SNVs detected in the trio-based and the duo-based analysis, according to an embodiment of the subject invention. X axis indicates the number of detected and the Y axis indicates the number of recurrent SNVs.

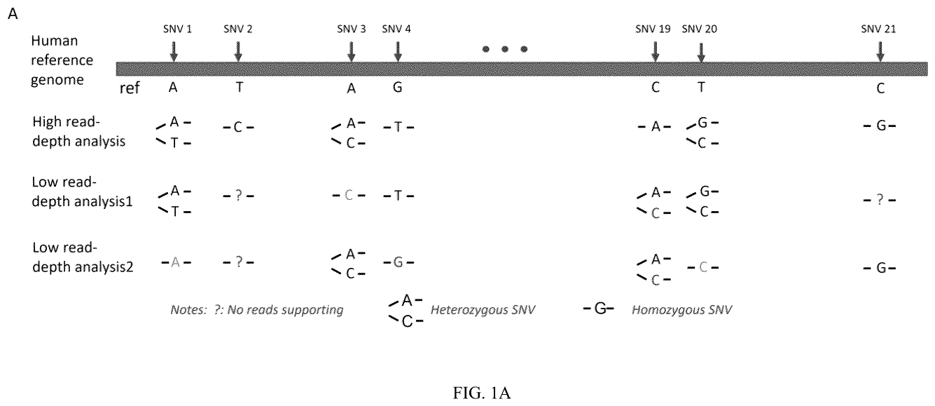

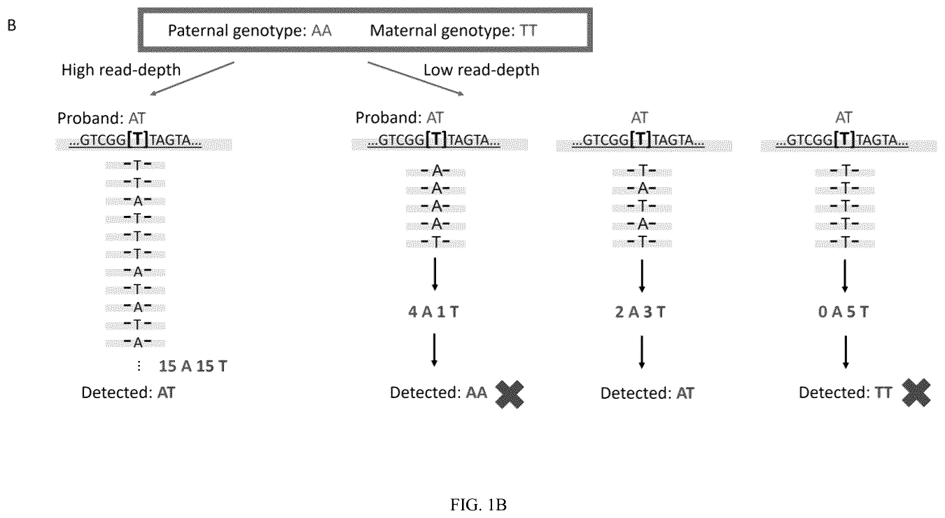

### DETAILED DISCLOSURE OF THE INVENTION

The principle of paternity/maternity testing is to affirm the paternity/maternity inclusion or exclusion according to the range of calculated paternity index. In the algorithm, an “inconsistent rate of base-type inheritance” between the proband and the presumed parent is used as paternity (or maternity) index for paternity (or maternity) confirmation.

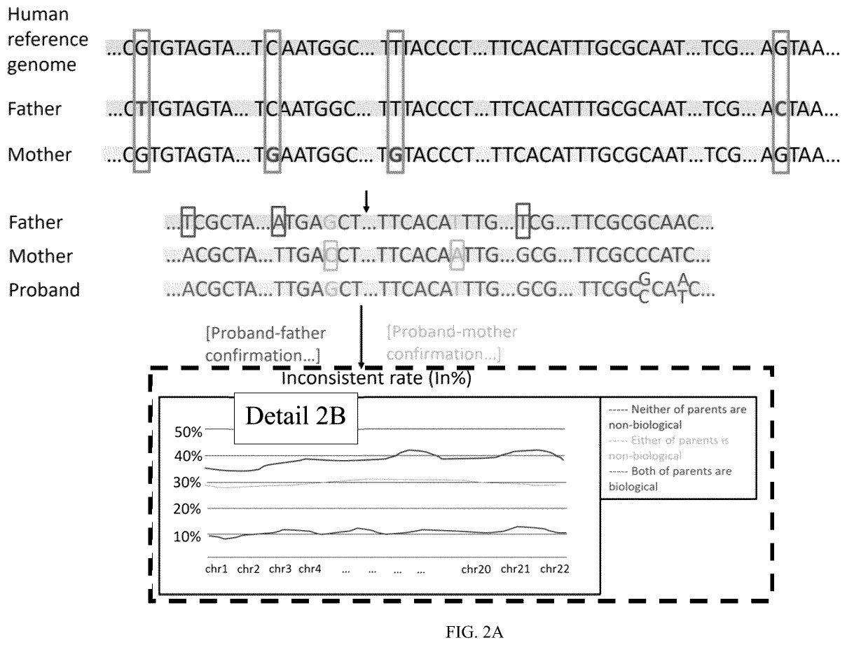

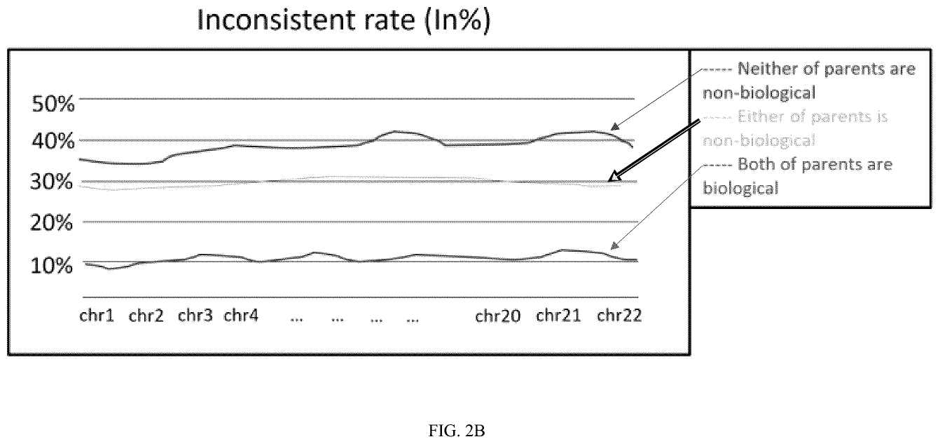

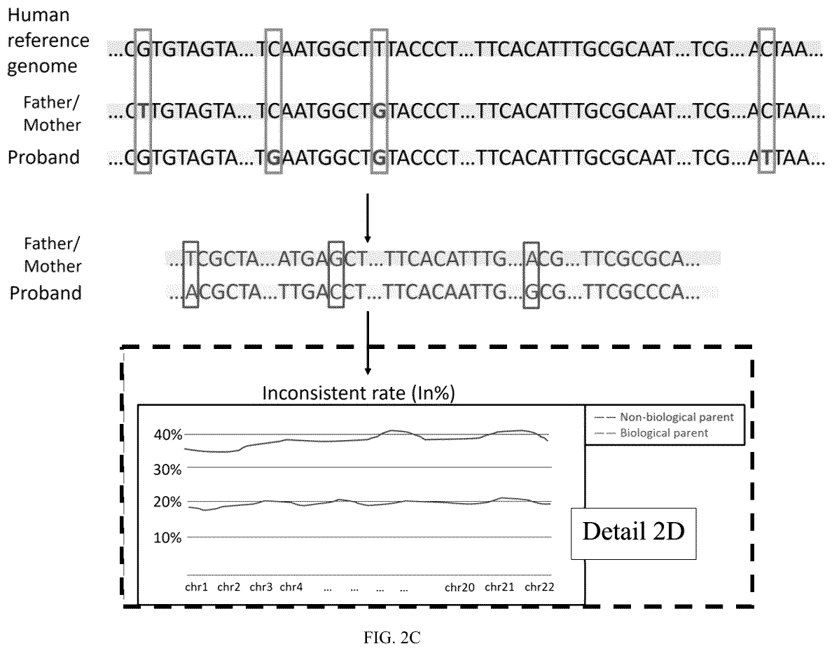

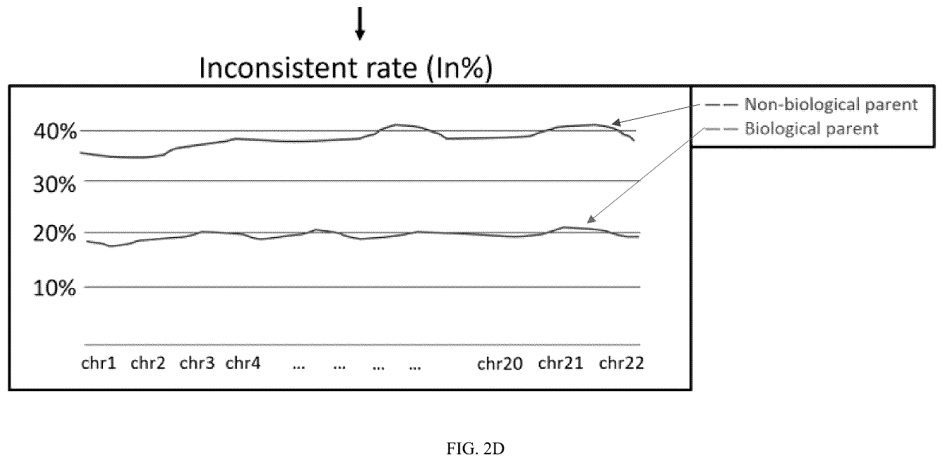

Two analytical models are presented in analytical pipeline: a duo mode and a trio mode. For the trio-based analysis mode, loci in which both parents were in homozygous for different genotypes were selected (for instance, a locus where the father was with homozygous A, whereas the mother was with homozygous T). In theory, the proband should carry a heterozygous AT genotype. However, in low-pass GS setting, proband can also show a homozygous genotype similar to one of parents (FIG. 2A). It might be due to: (a) one of the parents had a heterozygous genotype but mistakenly assigned as homozygous; (b) proband was called heterozygous but mistakenly assigned as homozygous; or (c) the genotype in one of the parents was resulted from systematic error(s). Lastly, the proband can carry a heterozygous genotype (e.g., AG) but one base type (i.e., G) was from neither parent. In addition to these false SNV calling events, the main reason for the inconsistency of base-type inheritance between the proband and the presumed parent(s) was non-paternity and/or non-maternity. Therefore, it is hypothesized that for paternity test (or maternity test), the inconsistent rate of base-type inheritance in a non-paternity (non-maternity) would be significantly higher than that in a biological family.

For the duo-based analytical mode, it is hypothesized that in a locus, if it was homozygous in the presumed father/mother, in the proband, it was heterozygous with one allele identical with that of the parent or homozygous that was the same as the submitted parent. However, in low-pass GS setting, it might be homozygous in the proband, but the genotype was different from the parent potentially because: (a) it was heterozygous in that parent but mistakenly assigned as homozygous; or (b) it was heterozygous in the proband but mistakenly assigned as homozygous; or (c) the genotype in one of them was resulted from systematic error(s). In addition to these false SNV calling events, the main reason for the inconsistent base-type inheritance between the proband and the presumed parent was non-paternity and/or non-maternity. Therefore, only those loci that both samples were in homozygous manner (green frames in FIG. 2C) were selected and rate that they were with different genotypes was calculated (blue frames in FIG. 2C).

In a first embodiment, a method of detecting parental inheritance of genotypes for paternity testing in biological samples from subjects is provided, comprising:
(i) aligning the sequence reads from low-pass genome sequencing of genomic DNA of biological samples to a human genome reference, and selecting and sorting sequence reads aligned to the human genome reference based on the aligned chromosome and genomic coordinates;
(ii) identifying single-nucleotide variants (SNVs) in the aligned sequence reads, wherein a single-nucleotide variant at each site has a mutant base type different from the base type at the corresponding site from the human genome reference;
(iii) identifying homozygous SNVs, diploid heterozygous SNVs from the SNVs identified in step (ii), wherein a homozygous SNV is defined based on the percentage of sequence reads supporting the mutant base type that is different from the base type at the corresponding site from the human genome reference being 100%, a diploid heterozygous SNV is defined based on the percentage of sequence reads supporting the mutant base type that is different from the base type at the corresponding site from the human genome reference being no less than 25% and no larger than 75%;
(iv) determining an inconsistent rate of base-type inheritance from the SNVs identified in step (iii) for the two analytical models, wherein for the trio-based analysis, (a) the number of loci that both parents were in homozygous manner but with different genotypes in the i-^{th} chromosome was denoted as Aⁱ; (b) among them, the number of SNVs that were homozygous in the proband but with different genotypes from the presumed father was denoted as pi (mi: for maternity test); (c) the inconsistent rate of paternal inheritance αi in chromosome i was calculated by formula (1);

$$α i = \frac{pi}{Ai} \qquad ( 1 )$$

(d) the paternity was determined by formula (2) using the average rate λ̅pat across all autosomal chromosomes;

$$\overline{λ} pat = \frac{{\sum }_{i = 1}^{n = 22} α i}{22} \qquad ( 2 )$$

The same process can be applied for maternity test. The inconsistent rate of maternal inheritance βi in chromosome i was calculated by the formula (3), while the maternity was determined by formula (4) using the average rate λ̅mat across all autosomal chromosomes;

$$β i = \frac{mi}{Ai} \qquad ( 3 )$$

$$\overline{λ} mat = \frac{{\sum }_{i = 1}^{n = 22} β i}{22} \qquad ( 4 )$$

For the duo-based analysis, (a) the number of homozygous SNVs in both proband and the presumed parent in chromosome i is denoted as Adi; (b) among them, the number of homozygous SNVs that were with different genotypes between the proband, and the presumed father was denoted as qi; (c) the inconsistent rate γi of paternal inheritance in chromosome i was calculated by formula (5):

$$γ i = \frac{qi}{Adi} \qquad ( 5 )$$

(d) the paternity was determined by formula (6) using the average rate λ̅pat across all autosomal chromosomes:

$$\overline{λ} pat = \frac{{\sum }_{i = 1}^{n = 22} γ i}{22} \qquad ( 6 )$$

Maternity was determined by the same method for the paternity determination.

As used herein, “subject,” “patient,” “individual” and grammatical equivalents thereof are used interchangeably and refer to, except where indicated, mammals, such as humans and non-human primates, as well as rabbits, felines, canines, rats, mice, squirrels, goats, pigs, deer, and other mammalian species. The term does not necessarily indicate that the subject has been diagnosed with a particular disease, but can refer to an individual under medical or veterinary supervision. In some embodiments, the subject is a female (pregnant or not pregnant), an infant, a male, or a subject with a need to confirm paternity/maternity. As understood by a person skilled in the art, paternity testing is useful in various settings, e.g., forensics, or to confirm parentage for prenatal or postnatal genetic diagnosis. Therefore, subject candidates or suitable biological samples can be determined by a person skilled in the art depending on the purpose for paternity testing.

The term “biological sample” or “sample from a subject” encompasses a variety of sample types obtained from an organism. The term encompasses bodily fluids such as blood, blood components, saliva, nasal mucous, serum, plasma, cerebrospinal fluid (CSF), urine and other liquid samples of biological origin, solid tissue biopsy, tissue cultures, peripheral blood, chorionic villus, amniotic fluid, cord blood, placental tissue, and tissue samples from organs, or supernatant taken from cultured patient cells. In the context of the present disclosure, the biological sample is typically a bodily fluid with detectable amounts of a subject's genome, e.g., a tissue sample, blood or a blood component (e.g., plasma or serum), saliva, oropharyngeal, nasopharyngeal, or a nasal secretion (mucous). The biological sample can be processed prior to assay, e.g., to remove cells or cellular debris. The term encompasses samples that have been manipulated after their procurement, such as by treatment with reagents, solubilization, sedimentation, or enrichment for certain components.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “isolated nucleic acid” molecule refers to a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated nucleic acid”.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

As used herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a nucleotide probe used in the method of this invention has at least 70% sequence identity, preferably 80%, 85%, 90%, 91%, 92%, 93%, 94%, 95%, 96%, 97%, 98%, 99%, or 100% identity, to a target sequence or complementary sequence thereof), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence.

Either single-end sequencing reads or paired-end sequencing reads (also referred to as “read-pairs”) are well known to a person skilled in the art, and can be suitably used in the present application.

The term “single-end sequencing” used herein refers to the sequencing technology in which a single end of a double stranded polynucleotide is sequenced using a specific primer binding site present on one end of the double stranded polynucleotide. The term “paired-end sequencing” used herein refers to the sequencing technology in which both ends of a double stranded polynucleotide are sequenced using specific primer binding sites present on each end of the double stranded polynucleotide, with more accurate read alignment and variants detection compared to single-end sequencing. Paired-end sequencing generates high-quality sequencing data, which is aligned using a computer software program to generate the sequence of the polynucleotide flanked by the two primer binding sites. Sequencing from both ends of a double stranded molecule allows high quality data from both ends of the double stranded molecule because sequencing from only one end of the molecule may cause the sequencing quality to deteriorate as longer sequencing reads are performed. Therefore, although both single-end sequencing and paired-end sequencing are available in the analysis, paired-end sequencing is the preferred type for analysis. A general description and the principle of paired-end sequencing is provided in Illumina Sequencing Technology, Illumina, Publication No. 770-2007-002, the contents of which are herein incorporated by reference in their entirety.

Non-limiting examples of the paired-end sequencing technology are provided by Illumina MiSeq™, Illumina MiSeqDx™, MGI Tech MGISEQ-2000, and Illumina MiSeqFGx™. Additional examples of the paired-end sequencing technology that can be used in the assays disclosed herein are known in the art and such embodiments are within the purview of the invention.

In certain embodiments, genomic DNA can be extracted from a biological sample. In certain embodiments, the amplified target genomic region can also be sequenced using techniques known in the art, for example, nanopore sequencing (Oxford Nanopore Technologies™), reversible dye-terminator sequencing (Illumina™) and Single Molecule Real-Time (SMRT) sequencing (PacBio™). Various sequencing instruments can be used for sequencing, such as using portable Nanopore Minion™ or benchtop machines, Nanopore Promethion™, PacBio Sequel™, MGI Tech MGISEQ-2000, or Illumina HiSeq™. The sequencing step can also be used for multiplex detection of several targets and/or polymorphism detection. Preferably, the sequencing of the amplified target genomic regions is performed on a high-throughput sequencer, such as an Illumina, PacBio, MGI Tech, or Nanopore device.

In certain embodiment, a sample can be subjected to small-insert size library construction (Cao, et al., 2022, which is hereby incorporated by reference in its entirety) or mate-pair library construction (Dong, et al., 2019, which is hereby incorporated by reference in its entirety). In certain embodiments, for small-insert size libraries, genomic DNA from each sample can be sheared into sizes of about 300 bp to about 500 bp, and then subjected to library construction, which can be performed using commercially available kits, such as, for example, using the MGIEasy FS DNA Library Prep kit, according to the manufacturer's protocol. In certain embodiments, each library (per sample) can be sequenced with single-end sequencing or paired-end sequencing with about a 100 bp to about a 150 bp read length for a read depth of at least about 1-fold, 2-fold, 3-fold, or 4-fold on a, for example, MGISEQ-2000 platform (MGI Tech Co., Ltd, Shenzhen, China). In certain embodiments, for mate-pair library construction, at least of 500 ng, about 1 μg, about 2 μg, or a greater amount of genomic DNA from each sample can be sheared into sizes of about 3000 bp to about 8000 bp by, for example, a HydroShear device (Digilab, Inc., Hopkinton, MA) and subjected for library construction through coupling Controlled Polymerizations by Adapter-Ligation (Dong, et al., 2019). A minimum of at least about 15 million read-pairs, about 30 million read-pairs, about 45 million read-pairs, or about 60 million read-pairs (about 100 bp to about 150 bp in length; equivalent to 4× read-depth) for each case (Dong, et al., 2021; Dong, et al., 2023) can be sequenced on a, for example, MGISEQ-2000 platform (MGI).

Library construction can be performed by extracting high quality DNA from blood samples sheared using the E220 Evolution focused-ultrasonicator (Covaris) to ˜5-kb in size. The sheared DNA will be purified with AmpureXP beads (Agencourt), followed by end-repair, A-tailing, and adaptor ligation. Adapter ligated DNA will be amplified using Pfu Turbo Cx enzyme (Agilent Technologies). The products will then be treated with USER (NEB) and T4 DNA ligase (Enzymatics) to form double-stranded circularized DNA. Nucleotide amount controlled nick translation (naCNT) will be performed using Bst DNA Polymerase, Full Length (NEB); Klenow fragment (Enzymatics). 3′branch ligation (3′BL) will be performed to ligate the adapter 2 (Ad2) to the 3′-end of the naCNT products. ttCPE (time and temperature-controlled primer extension) will be performed and will be ligated to the 5′-end of Ad2 and further amplified using Pfu Turbo Cx. Single-stranded circularized DNA will be generated by denaturation of the library and ligation. DNA nanoballs will be formed through rolling chain amplification for sequencing on the MGISEQ-2000 platform (MGI Technology Ltd. Co., Shenzhen, China) (see, for example, Zirui Dong and others, Development of coupling controlled polymerizations by adapter-ligation in mate-pair sequencing for detection of various genomic variants in one single assay, DNA Research, Volume 26, Issue 4, August 2019, Pages 313-325, which is hereby incorporated by reference in its entirety).

As compared with GS requiring sequencing, the low-pass GS in the present application can have a lower read depth, e.g., between about 1-fold to about 15-folds. For example, 1-fold.

Suitable human genome reference for alignment step can be selected by a person skilled in the art. In a particular embodiment, the human genome reference is hg19/GRCh37, hg38/GRCh38, T2T-CHM13v2.0.

Suitable human genome reference for alignment step can also be selected by a person skilled in the art, including, but not limited to, Short Oligonucleotide Alignment Program 2 (SOAP2) or Burrows-Wheeler Aligner (BWA) and Bowtie2. Default setting can be adopted.

In some embodiments, step (ii) further includes removing sequence reads due to polymerase chain reaction (PCR) duplication.

In some embodiments, step (iii) further includes discarding a site as described below:
(a) a minimal read-depth of the site is determined by the minimal read-depth of the biological sample;
(b) a maximum read-depth of the site is determined by the maximal read-depth of the biological sample; or
(c) a site where no sequence read supports a mutant base type.

In some embodiments, paternity or maternity determination in step (v) was determined by increased inconsistent rate over the cutoff for the paternity or maternity test. A process from evaluating the precise cutoff for the paternity test to parentage determination in case samples is described below.
(i) Data preprocessing
   10 trios with confirmed biological relationship were selected and data from different families were randomized to form non-paternity (or non-maternity) families. To determine the optimal sequencing parameters for paternity testing including read-length, read-depth, library construction (small-insert library or mate-pair library), and sequencing-mode (paired-end or single-end), read1 was used from the paired-end sequencing data as single-end sequencing data, while 150 bp reads were trimmed into 100 bp to serve as sequencing data with shorter read-length. Down-sampling of the sequencing data was performed based on the general read-depth (0.5, 1, 2, 3 and 4-fold).
(ii) Alignment
   For each sample, single-end reads or paired-end reads are subjected for alignment to the human genome reference (such as GRCh37/hg19, GRCh38/hg38, or T2T-CHM13v2.0) by a suitable alignment software (e.g., Short Oligonucleotide Alignment Program 2 (SOAP2), Burrows-Wheeler Aligner (BWA), or Bowtie2) with default settings. All the reads/read-pairs aligned to the human genome reference are selected, and sorted based on the aligned chromosome and coordinates, followed by removal of reads/read-pairs due to polymerase chain reaction (PCR) duplication by, for example, SAMtools program. PCR duplicates can be removed if multiple read pairs have identical external coordinates, only retaining the pair with highest mapping quality (see, for example, the description of the SAMtools program at worldwide website: manpages.ubuntu.com/manpages/kinetic/en/man1/samtools-rmdup.1.html, which is hereby incorporated by reference in its entirety). The remaining reads/read-pairs are named as processed reads/read-pairs and are subjected for further analysis.
(iii) Putative SNVs calling
   The processed reads/read-pairs from step (ii) are used as input for identifying the alignment result in each coordinate by MPileup module from SAMtools. From each site, the aligned information can present as:
      a. “.” is with consistent base type as human genome reference and the aligned strand is plus or “+”;
      b. “,” is with consistent base type as human genome reference and the aligned strand is minus or “−”;
      c. “A” (using base type “A” as example) is with mutant base type different from the base type from human genome reference and the aligned strand is plus or “+”;
      d. “a” (using base type “A” as example) is with mutant base type different from the base type from human genome reference and the aligned strand is minus or “−”.
   From each site, the chromosome, coordinate, base type in reference, and the aligned information are subjected for putative SNVs detection and the following sites can be discarded:
   a. A minimal read-depth of each “putative” site is determined by the minimal read-depth of the particular sample. A SNV is defined if there are 5 to 20 reads covered that locus and over two reads supporting a mutant base type. For example, when there is only 3-fold for a case, those sites with read-depth <3 can be discarded. In addition, given the sequencing read-depth is following a normal distribution, those sites with extremely higher read-depth such as >mean+3SD (standard deviations) can be also discarded since they are mostly likely resulted from systematic errors; or
   b. No read supporting a mutant base type;
(iv) Identifying homozygous SNVs, diploid heterozygous SNVs
   A homozygous SNV is defined based on the percentage of sequence reads supporting the mutant base type different from the base type at the corresponding site from the human genome reference being 100%, and a diploid heterozygous SNV is defined based on the percentage of sequence reads supporting the mutant base type different from the base type at the corresponding site from the human genome reference being no less than 25% and no larger than 75%;
(v) Inconsistent rate calculation of base-type inheritance from the SNVs
   For the trio-based analysis, (a) the number of loci that both parents were in homozygous manner but with different genotypes in the i-^{th} chromosome was denoted as Aⁱ; (b) among them, the number of SNVs that were homozygous in the proband but with different genotypes from the presumed father was denoted as pi (mi: for maternity test); (c) the inconsistent rate of paternal inheritance αi in chromosome i was calculated by the formula (1); (d) the paternity was determined by formula (2) using the average rate λ̅pat across all autosomal chromosomes; the same process can be applied for maternity test, the inconsistent rate of maternal inheritance βi in chromosome i was calculated by the formula (3), while the maternity was determined by formula (4) using the average rate λ̅mat across all autosomal chromosomes. For the duo-based analysis, (a) the number of homozygous SNVs in both proband and the presumed parent in chromosome i is denoted as Adi; (b) among them, the number of homozygous SNVs that were with different genotypes between the proband and the presumed father was denoted as qi; (c) the inconsistent rate γi of paternal inheritance in chromosome i was calculated by formula (5); (d) the paternity was determined by formula (6) using the average rate λ̅pat across all autosomal chromosomes; the maternity was determined with the same method for the paternity determination.
(vi) Parameter selection
   The trio-based and the duo-based modes were performed for each biological and putative families with the same analytical parameters to calculate the inconsistent rates of paternal/maternal inheritance for comparison respectively. The optimal parameter was determined by substantial difference of the inconsistent rate of parental inheritance between biological and non-biological groups, and the consideration of efficiency and cost of the analysis.
(vii) Cutoff determination and evaluation
   With the parameter selected in step (vi), the average and SD of the inconsistent rate of parental inheritance among the biological families and the simulated non-paternity/non-maternity families were calculated to determine the cutoff for two modes of analysis. Additional 170 trios among different methods of library constructions and different sequencing platforms were randomly selected to validate the performance.

In a second embodiment, a computer system for calculating inconsistent rate of base-type inheritance for paternity testing in biological samples from subjects is provided, comprising a processor and a memory storing a plurality of instructions, wherein the processor, upon processing the instructions, is configured to perform the following steps:
(i) aligning sequence reads from low-pass genome sequencing of genomic DNA of biological samples to a human genome reference, and selecting and sorting sequence reads aligned to the human genome reference based on the aligned chromosome and genomic coordinates;
(ii) identifying single-nucleotide variants (SNVs) in the aligned sequence reads, wherein a single-nucleotide variant at each site has a mutant base type different from the base type at the corresponding site from the human genome reference;
(iii) identifying homozygous SNVs and/or diploid heterozygous SNVs from the SNVs identified in step (ii), wherein a homozygous SNV is defined based on the percentage of sequence reads supporting the mutant base type that is different from the base type at the corresponding site from the human genome reference being 100%, a diploid heterozygous SNV is defined based on the percentage of sequence reads supporting the mutant base type that is different from the base type at the corresponding site from the human genome reference being no less than 25% and no larger than 75%;
(iv) determining an inconsistent rate of base-type inheritance from the SNVs identified in step (iii) for two analytical models, wherein for trio-based analysis, (a) the number of loci that both parents were in homozygous manner but with different genotypes in the i-^{th} chromosome was denoted as Aⁱ; (b) among them, the number of SNVs that were homozygous in the proband but with different genotypes from the presumed father was denoted as pi (mi: for maternity test); (c) the inconsistent rate of paternal inheritance αi in chromosome i was calculated by the formula (1); (d) the paternity was determined by formula (2) using the average rate λ̅pat across all autosomal chromosomes; the same process can be applied for maternity test, and the inconsistent rate of maternal inheritance βi in chromosome i was calculated by formula (3), while the maternity was determined by formula (4) using the average rate λ̅mat across all autosomal chromosomes. For the duo-based analysis, (a) the number of homozygous SNVs in both proband and the presumed parent in chromosome i is denoted as Adi; (b) among them, the number of homozygous SNVs that were with different genotypes between the proband and the presumed father was denoted as qi; (c) the inconsistent rate γi of paternal inheritance in chromosome i was calculated by the formula (5); (d) the paternity was determined by formula (6) using the average rate λ̅pat across all autosomal chromosomes; and the maternity was determined by the same method applied for the paternity determination.

In a third embodiment, a computer readable medium storing a plurality of instructions is provided, wherein the plurality of instructions, upon executed by one or more processors, perform an operation including the following steps:
(i) aligning sequence reads from low-pass genome sequencing of genomic DNA of biological samples to a human genome reference, and selecting and sorting sequence reads aligned to the human genome reference based on the aligned chromosome and genomic coordinates;
(ii) identifying single-nucleotide variants (SNVs) in the aligned sequence reads, wherein a single-nucleotide variant at each site has a mutant base type different from the base type at the corresponding site from the human genome reference;
(iii) identifying homozygous SNVs and/or diploid heterozygous SNVs from the SNVs identified in step (ii), wherein a homozygous SNV is defined based on the percentage of sequence reads supporting the mutant base type different from the base type at the corresponding site from the human genome reference being 100%, a diploid heterozygous SNV is defined based on the percentage of sequence reads supporting the mutant base type different from the base type at the corresponding site from the human genome reference being no less than 25% and no larger than 75%;
(iv) determining an inconsistent rate of base-type inheritance from the SNVs identified in step (iii) for two analytical models, wherein for trio-based analysis, (a) the number of loci that both parents were in homozygous manner but with different genotypes in the i-^{th} chromosome was denoted as Aⁱ; (b) among them, the number of SNVs that were homozygous in the proband but with different genotypes from the presumed father was denoted as pi (mi: for maternity test); (c) the inconsistent rate of paternal inheritance αi in chromosome i was calculated as the formula (1); (d) the paternity was determined by formula (2) using the average rate λ̅pat across all autosomal chromosomes; the same process can be applied for the maternity test, the inconsistent rate of maternal inheritance βi in chromosome i was calculated by the formula (3), while the maternity was determined by formula (4) using the average rate λ̅mat across all autosomal chromosomes. For the duo-based analysis, (a) the number of homozygous SNVs in both proband and the presumed parent in chromosome i is denoted as Adi; (b) among them, the number of homozygous SNVs that were with different genotypes between the proband and the presumed father was denoted as qi; (c) the inconsistent rate γi of paternal inheritance in chromosome i was calculated by the formula (5); (d) the paternity was determined by formula (6) using the average rate λ̅pat across all autosomal chromosomes. The maternity was determined by the same method applied for paternity determination.

The features or embodiments described in a first embodiment can be applied to or combined into a second or a third embodiment.

Embodiments of the subject invention address the technical problem of determining paternity and/or maternity is costly by high read-depth genome sequencing, and is laborious by other methods such as quantitative fluorescent PCR with short tandem repeat markers.

This problem is addressed by providing advanced analysis of low-pass genome sequencing reads, determining an inconsistent rate of base-type inheritance of single-nucleotide variants (SNVs) between the proband and the presumed parent(s), and applying a duo based analytical framework, a trio based analytical framework, or both to determine maternity and/or paternity.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention. It should be understood that any of the embodiments of the present invention can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at the same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual embodiment, or specific combinations of these individual embodiments.

The above description of example embodiments of the invention has been presented for the purposes of illustration and description. It is not intended to be exhaustive or to limit the invention to the precise form described, and many modifications and variations are possible in light of the teaching above.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Additionally, details of any specific embodiment may not always be present in variations of that embodiment or may be added to other embodiments.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

A recitation of “a”, “an” or “the” is intended to mean “one or more” unless specifically indicated to the contrary. The use of “or” is intended to mean an “inclusive or,” and not an “exclusive or” unless specifically indicated to the contrary.

All patents, patent applications, publications, and descriptions mentioned herein are incorporated by reference in their entirety for all purposes. None is admitted to be prior art.

### Materials and Methods

All patents, patent applications, provisional applications, and publications referred to or cited herein are incorporated by reference in their entirety, including all figures and tables, to the extent they are not inconsistent with the explicit teachings of this specification.

The following are examples that illustrate procedures for practicing the invention. These examples should not be construed as limiting. All percentages are by weight and all solvent mixture proportions are by volume unless otherwise noted.

### Example 1

Informed written consent for sample storage and genetic analyses was obtained from each participant. In this study, there were 130 products of conception, prenatal (chorionic villi, or amniotic fluid) and postnatal samples with the presumed parents recruited.

DNA preparation for low-pass GS was completed as follows. Genomic DNA was extracted with DNeasy Blood & Tissue Kit (cat. number/ID: 69506, Qiagen, Hilden, Germany) and treated with RNase (Qiagen, Hilden, Germany). DNA was subsequently quantified with the Qubit dsDNA HS Assay Kit (Invitrogen, Carlsbad, CA, USA) and DNA integrity was assessed by gel electrophoresis. All samples passing QC (>500 ng; OD260/OD280>1.8; OD260/OD230>1.5) were subsequently prepared for library construction in low-pass GS with two library construction methods.

Low-pass GS was completed as follows. The inventors selected 10 trios with confirmed biological relationship for low-pass GS according to an embodiment of the subject invention. Five trios (15 samples) were subjected for small-insert size library construction (Cao, et al., 2022), and the other five were subjected for mate-pair library construction (Dong, et al., 2019). For small-insert size libraries, genomic DNA from each sample was sheared with the Covaris E220 Evolution Focused-Ultrasonicator (Covaris, Inc., Woburn, MA) into sizes of 300-500 bp, and then subjected to library construction using the MGIEasy FS DNA Library Prep kit according to the manufacturer's protocol. Each library (per sample) was sequenced with paired-end 150 bp for a read depth of ˜4-fold on an MGISEQ-2000 platform (MGI Tech Co., Ltd, Shenzhen, China). For mate-pair library construction, 1 μg of genomic DNA from each sample was sheared (3˜8 kb) by a HydroShear device (Digilab, Inc., Hopkinton, MA) and subjected for library construction following reported methods (Dong, et al., 2019). A minimum of 60 million read-pairs (100 bp in length; equivalent to 4× read-depth) for each case (Dong, et al., 2021; Dong, et al., 2023) on an MGISEQ-2000 platform (MGI).

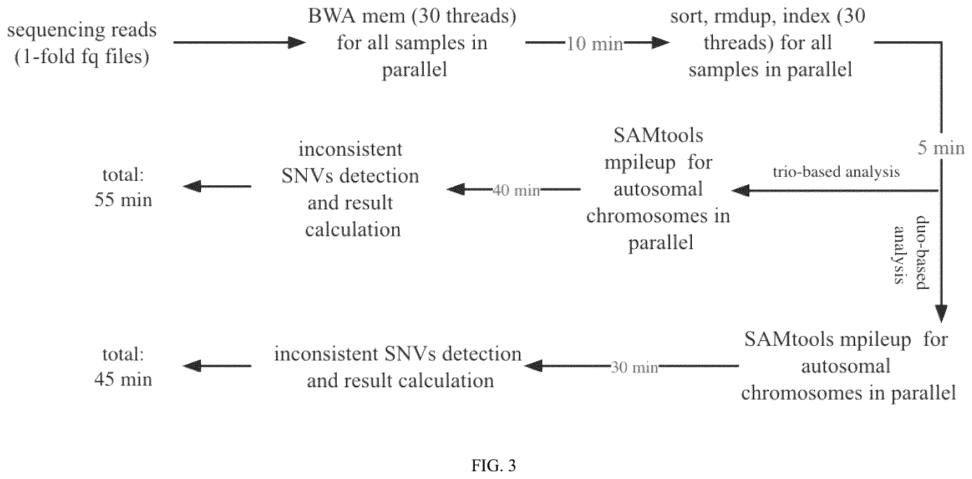

LpPat analysis for determination of the parental inheritance according to an embodiment of the subject invention was completed as follows. After data QC assessment, the read/read-pairs were aligned to the human reference genome (GRCh37) by Burrows-Wheeler Aligner (BWA)(Li and Durbin, 2009) with mem module. With SAMtools (Li, et al., 2009), the alignment file was then sorted by aligned chromosomes and locations, and the reads that were likely generated from PCR duplication were removed. It was then reformatted by the Mpileup module from SAMtools to calculate the coverage and to determine the genotype of each genomic location. Loci with read(s) supporting a mutant base type were selected for further analysis. A SNV was defined if there were 5 to 20 reads covered that locus and over two reads supporting a mutant base type (Dong, et al., 2021). The genotype of this SNV was defined as homozygous if 100% of reads were supporting the mutant base type, whereas a heterozygous SNV was defined as 25 to 75% of reads supporting the mutant base type. Two modes of analysis were provided by an embodiment of the subject invention, referred to herein as LpPat. Calculation of the inconsistent rate of paternal/maternal inheritance was performed as described above (FIG. 3), and the paternity/maternity was identified by comparing the corresponding thresholds.

Data simulation was completed as follows. To determine the precise cutoff for the paternity test, parental data from different families were randomized to form non-paternity (or non-maternity) families among the 10 trios. In addition, to determine the optimal sequencing parameters for paternity testing (e.g., read-length, read-depth, library construction, and sequencing-mode (paired-end or single-end)), the inventors used read1 from the paired-end sequencing data as single-end sequencing data, while 150 bp reads were trimmed into 100 bp to serve as sequencing data with shorter read-length. Down-sampling of the sequencing data based on the general read-depth (0.5, 1, 2, 3 and 4-fold) was performed.

To evaluate the accuracy of using the optimal parameters for paternity detection, trio-based and duo-based analyses were performed on another 120 clinical trios sequenced in MGIseq-2000 platform (MGI) including 100 trios sequenced with small-insert libraries and 20 trios sequenced with mate-pair libraries. In addition, 50 trios sequenced in NovaSeq 6000 System (Illumina, San Diego, CA, USA) with small-insert libraries were also randomly selected from the 1000 Genomes Project (1KGP) (Byrska-Bishop, et al., 2022) for further analysis (Table 1). The GS data in CRAM format were downloaded from the 1KGP, and converted into Fastq format. To compare the performance with the same sequencing setting among different datasets, for the data sequenced with small-insert libraries (both MGISeq-2000 or NovaSeq), 150 bp reads were trimmed into 100 bp and each sample was down-sampled at 1-fold read-depth.

Distribution of SNVs was investigated as follows. To investigate whether detected SNVs were recurrent among all analyzed trios, the distributions of these SNVs among all 180 trios in biological and simulated non-biological families were compared.

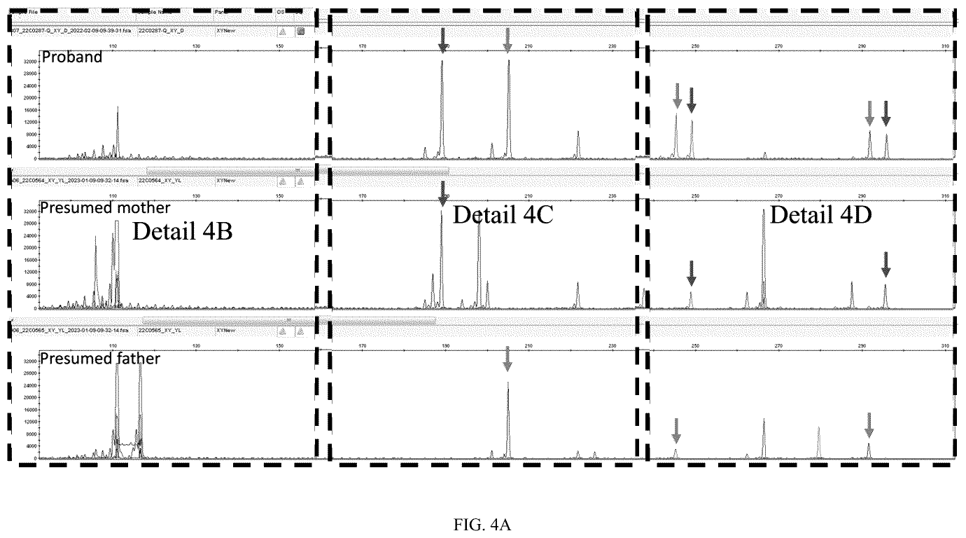

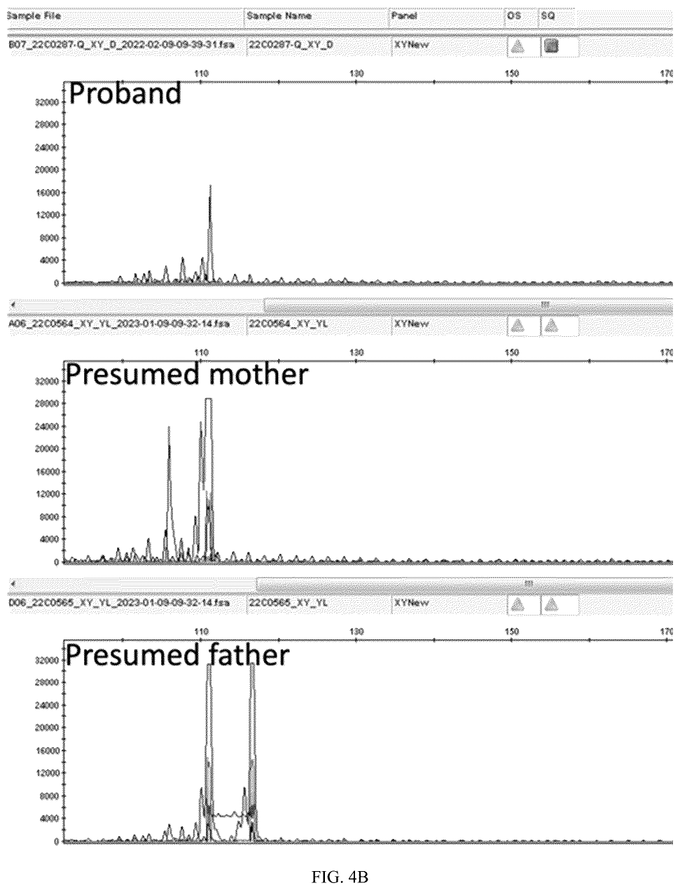

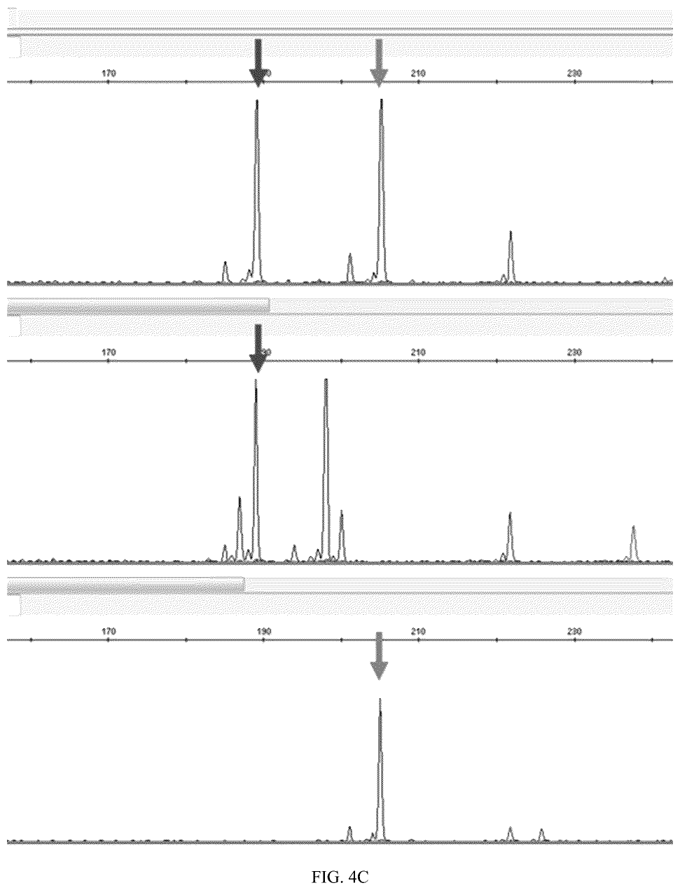

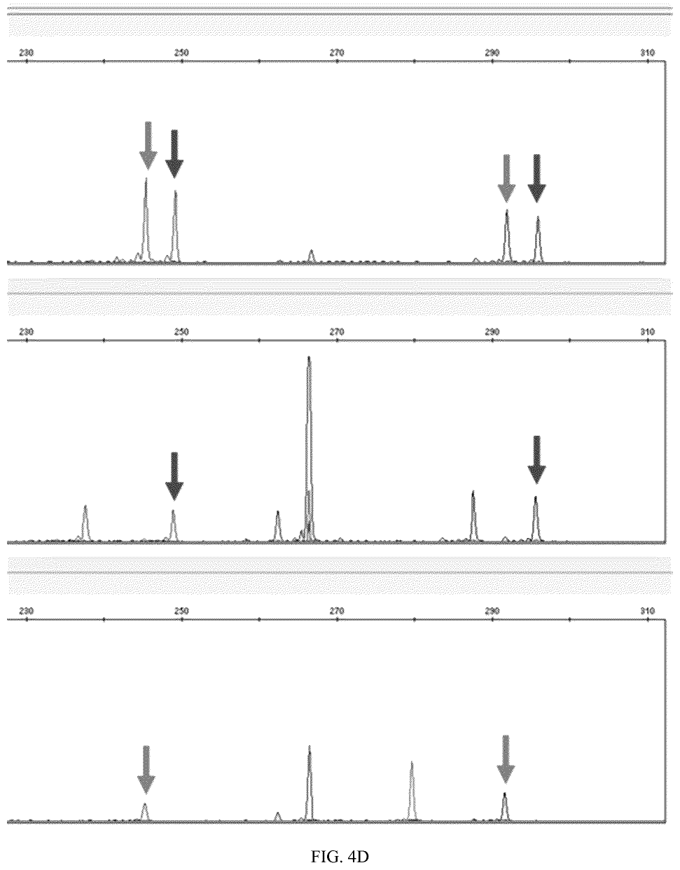

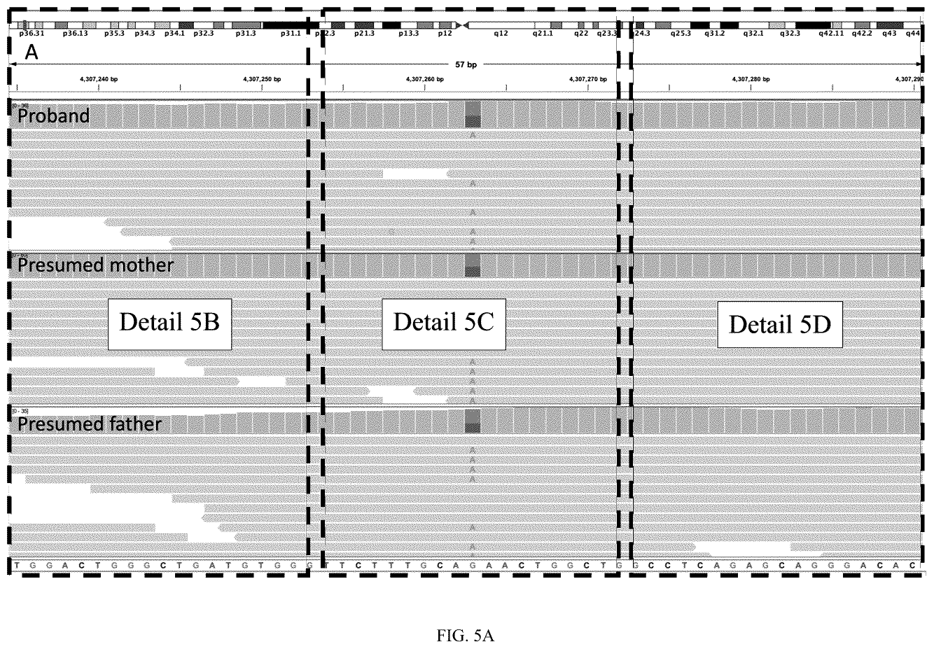

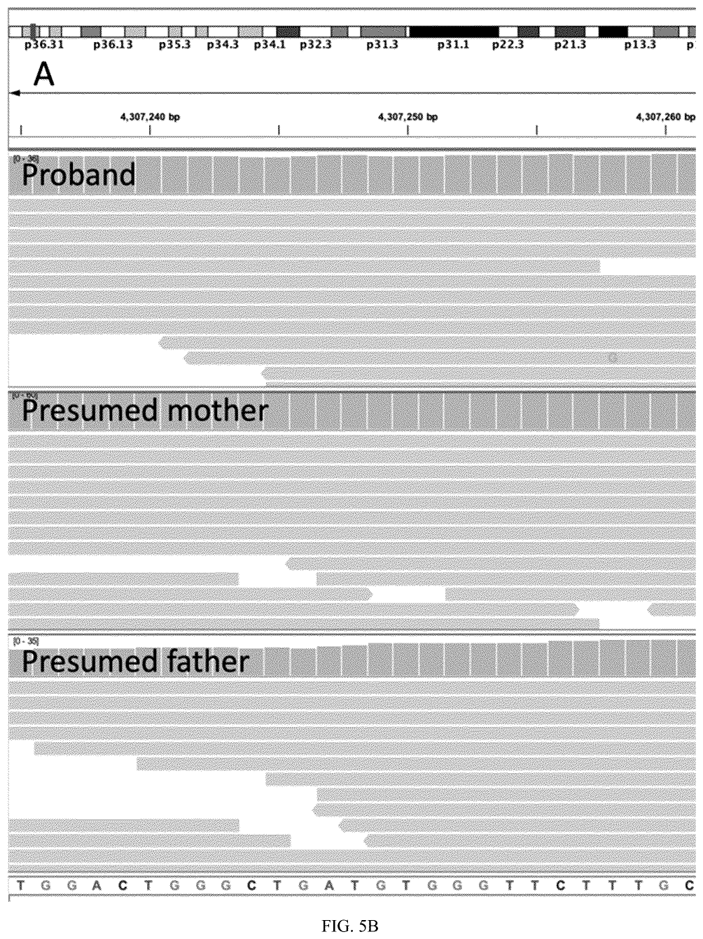

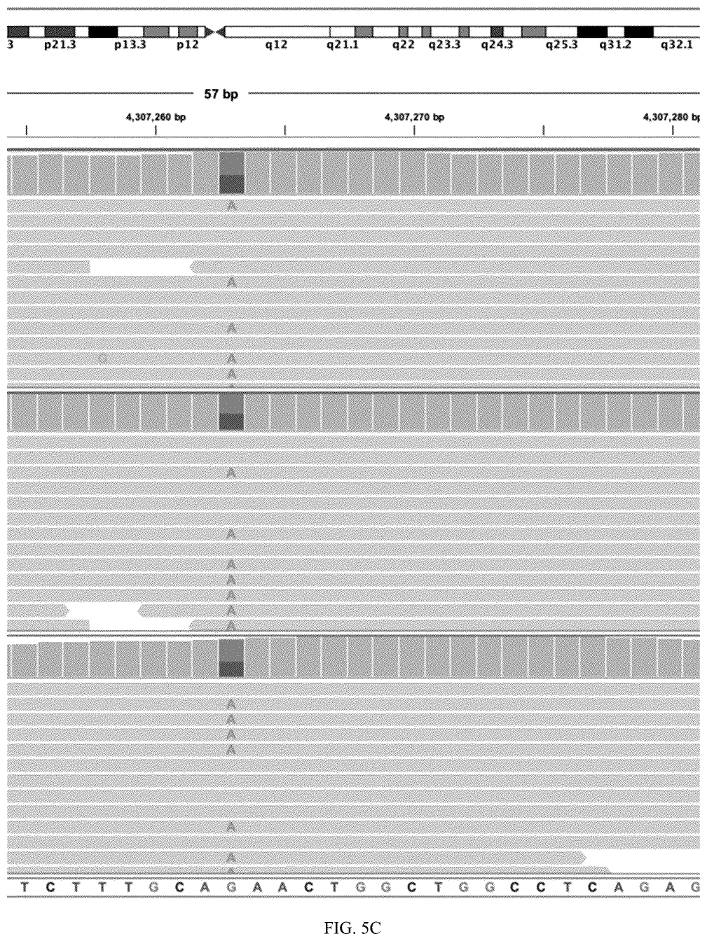

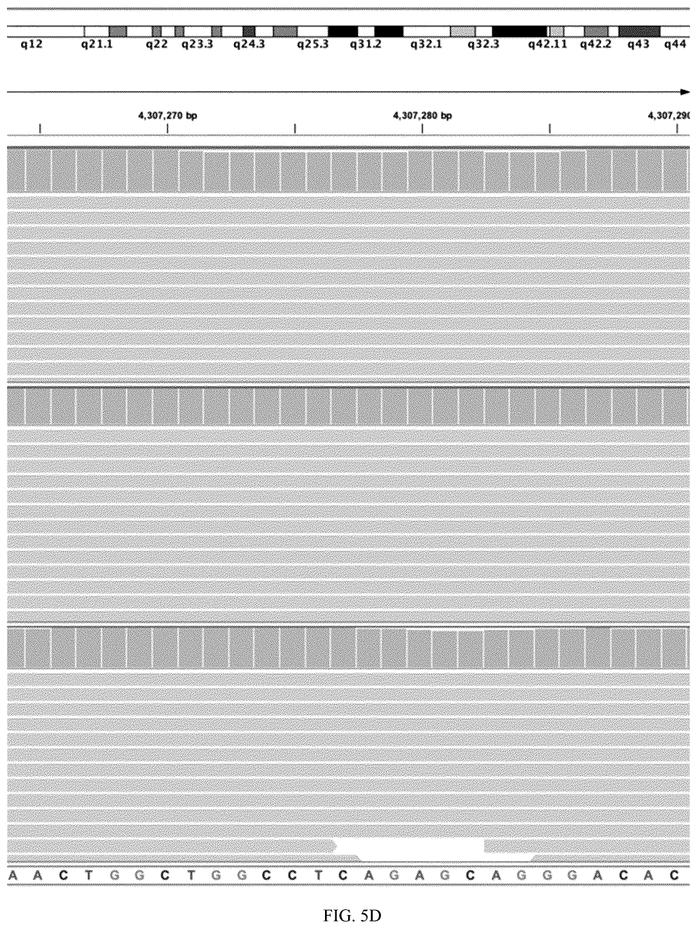

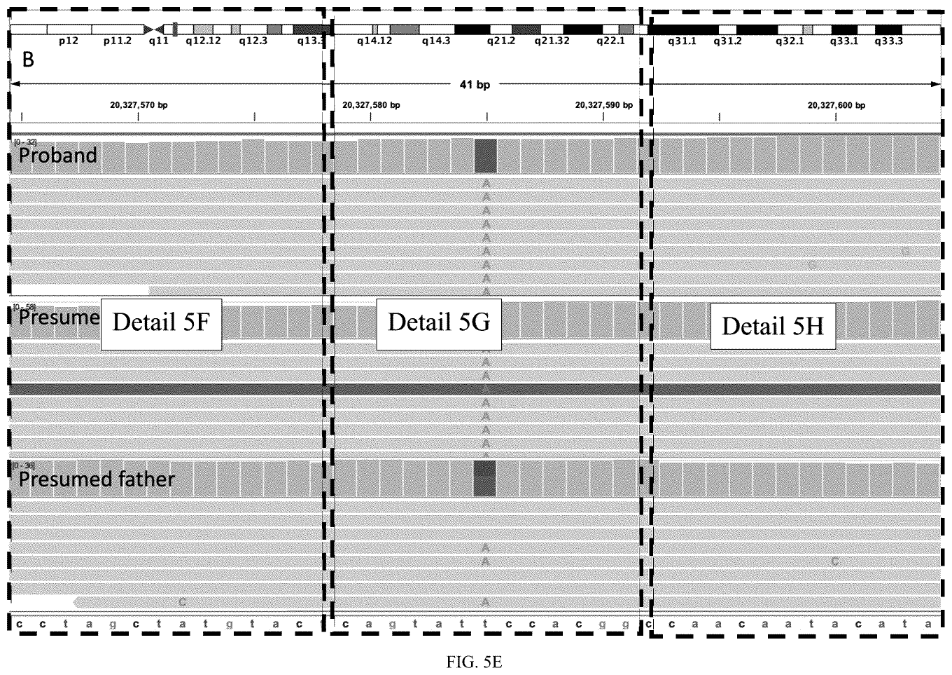

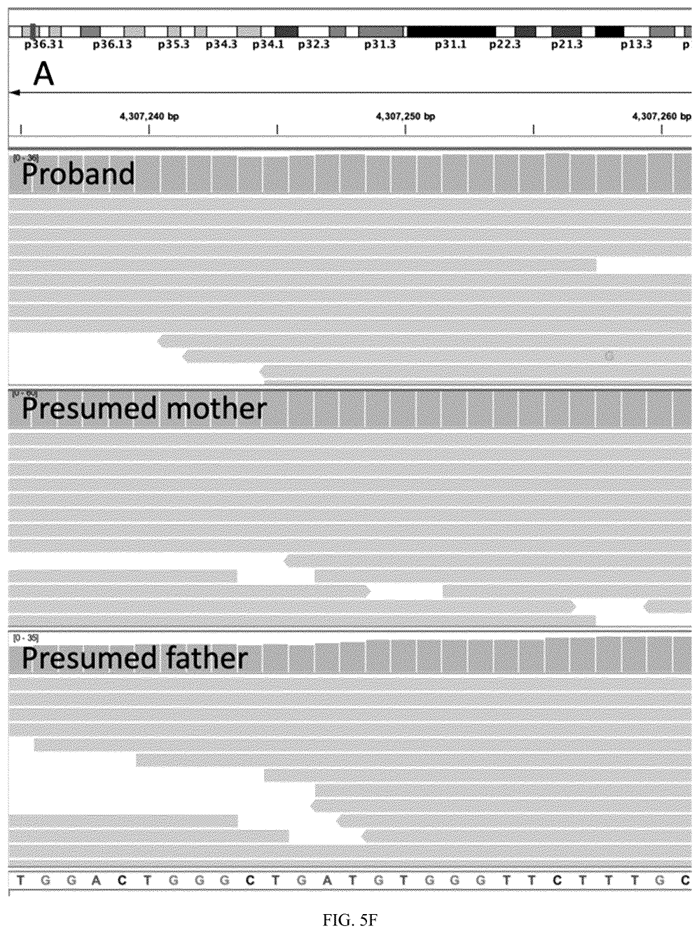

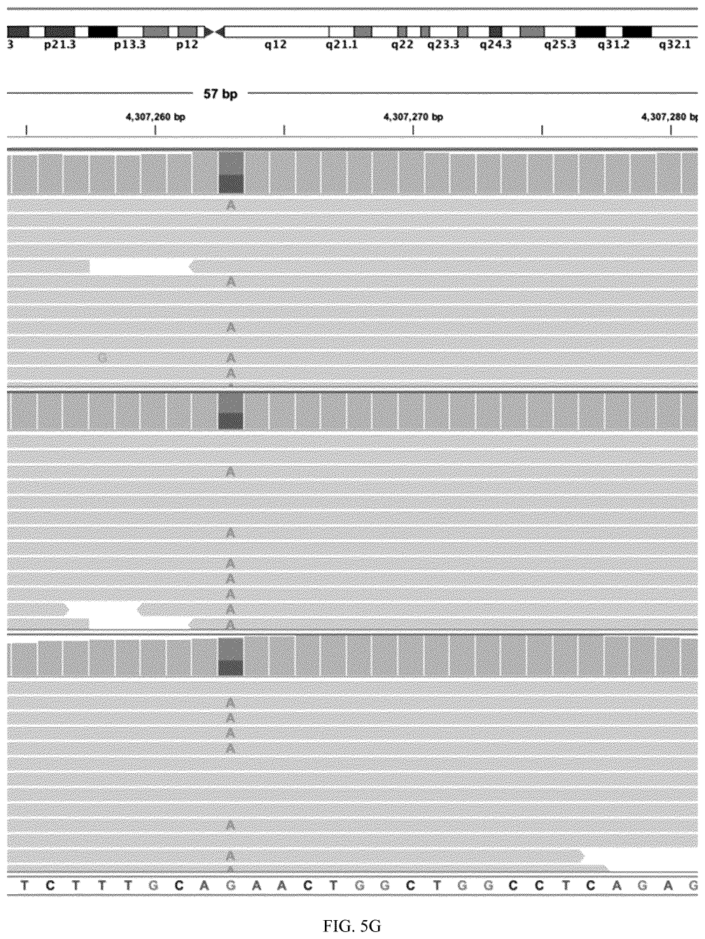

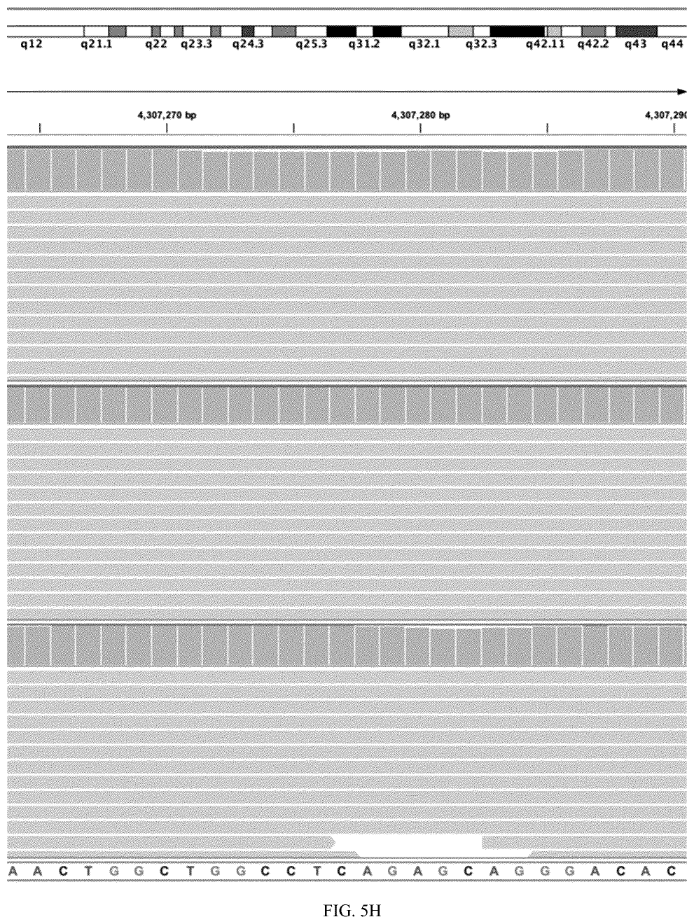

Verification of parental inheritance was completed as follows. For the clinical samples in Phase I (10 trios) and Phase II (120 trios), parental inheritance was confirmed by quantitative fluorescence polymerase chain reaction (QF-PCR) with 100 ng DNA from each sample by using short tandem repeat (STR) markers located on chromosomes 13, 18, 21, X, and Y (FIGS. 4A-4D)(Wang, et al., 2020). For the 50 trios downloaded from the 1000 Genomes Project, the inventors utilized the genotypes from each family member identified via high read-depth GS for the confirmation (FIGS. 5A-5H) among those SNPs commonly used for paternity test (Schwark, et al., 2012).

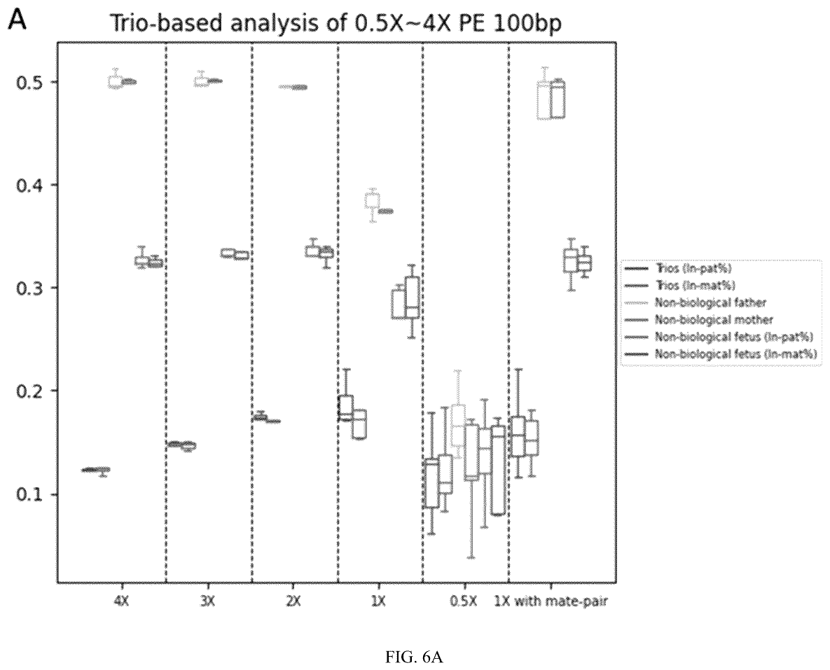

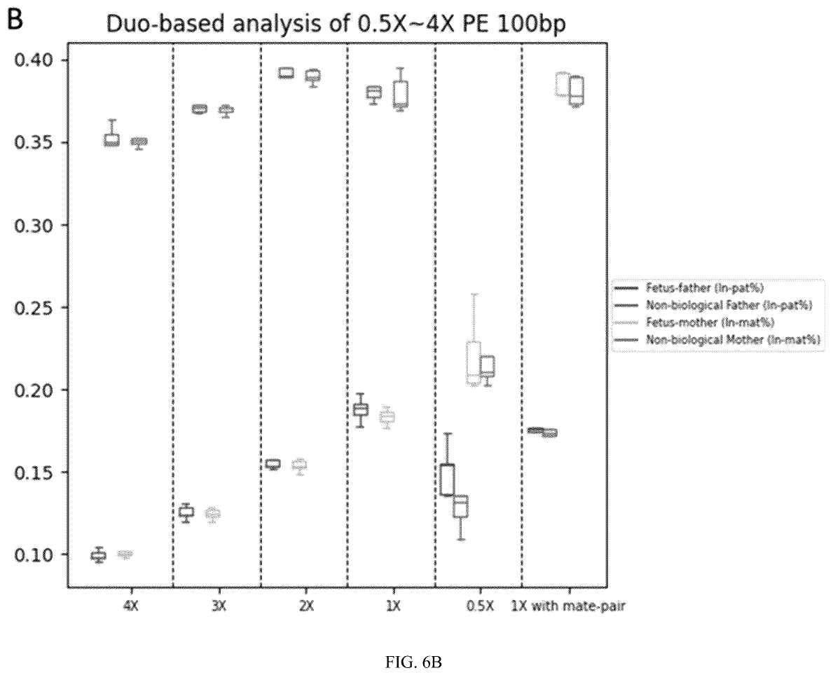

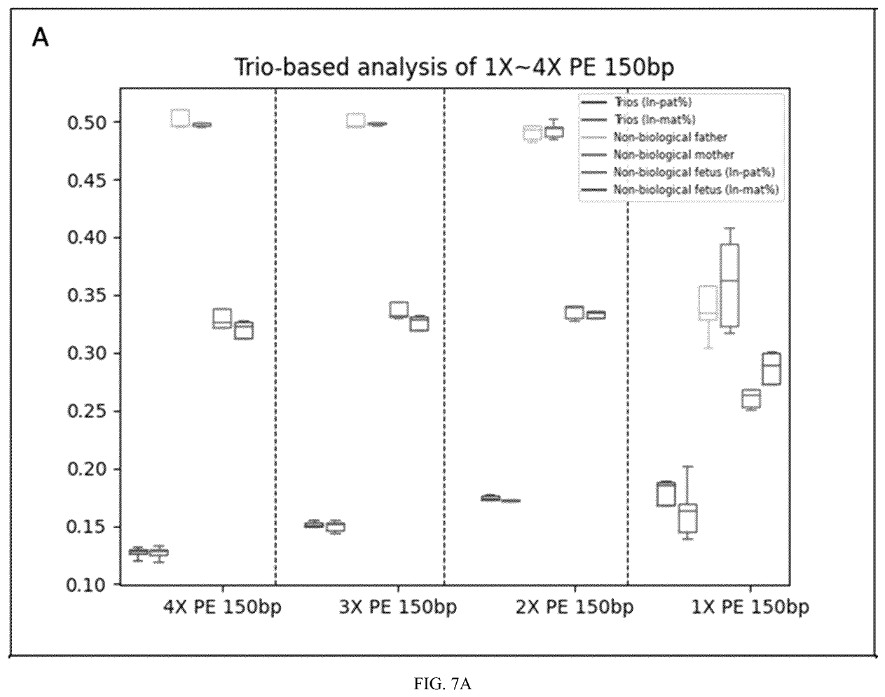

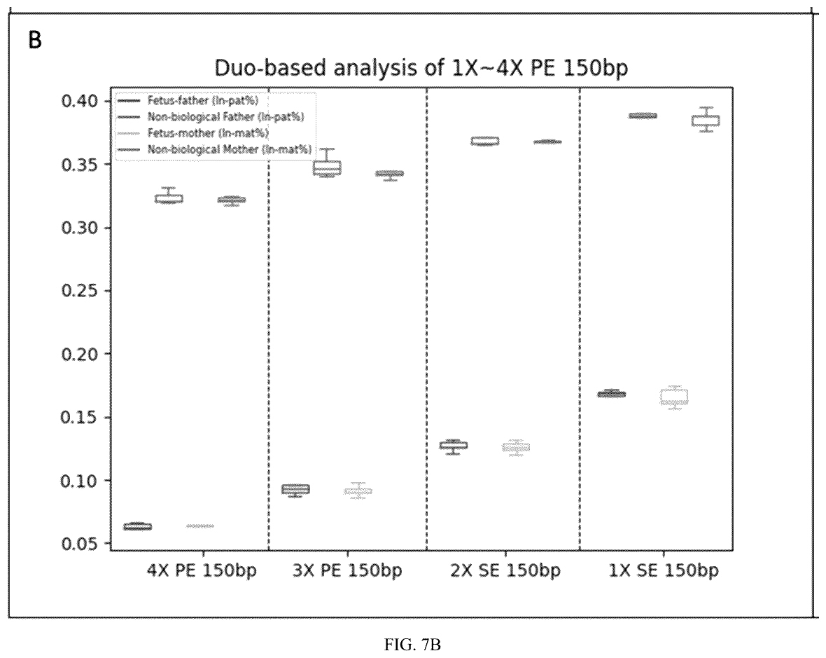

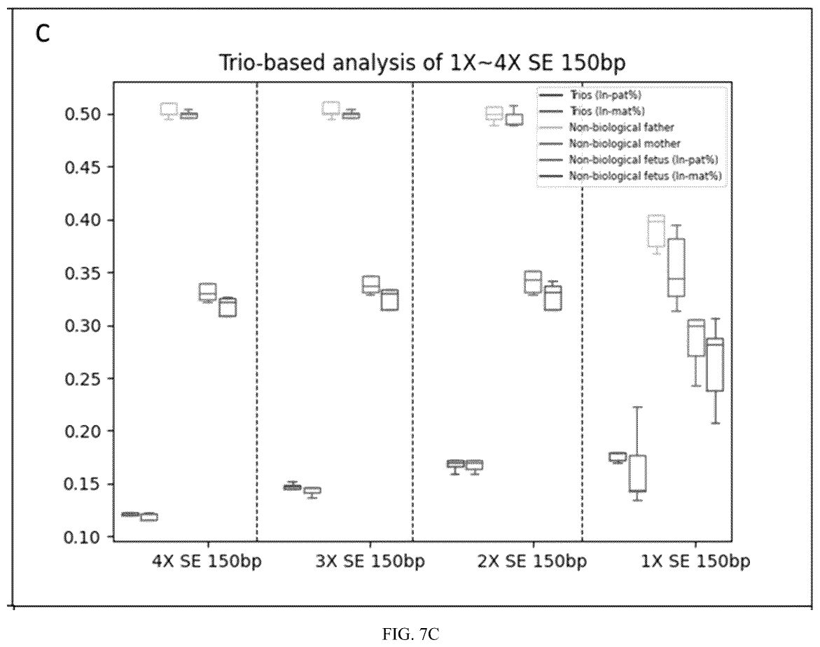

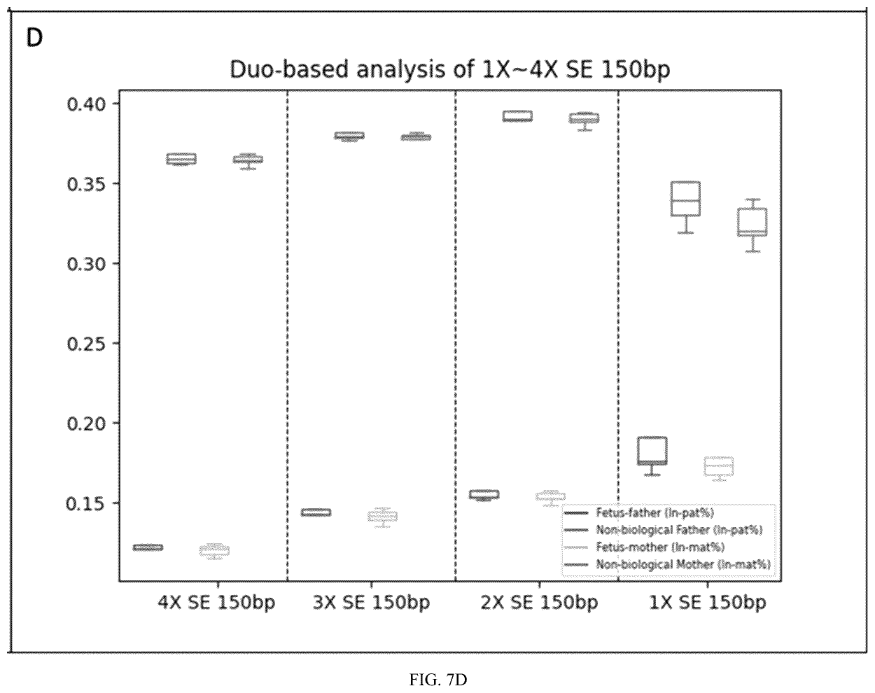

Results included establishment of optimal parameters for LpPat according to an embodiment of the subject invention, as follows. The inventors selected 10 trios with confirmed paternity and maternity, and performed low-pass GS with two types of library constructions. In addition, data simulation was performed for each sample to generate different sets of low-pass GS data with consistent sequencing parameters (e.g., read-lengths, read-depths, and sequencing modes) among the family members by down-sampling the sequencing data (e.g., 0.5, 1, 2, 3, 4-fold). In addition, the inventors randomly assigned the paternal/maternal samples for each family to form a non-paternity and/or non-maternity family. Trio-based and duo-based modes were performed for each family with the same analytical parameters to calculate the inconsistent rates of paternal/maternal inheritance for comparison (FIGS. 6A-6B and 7A-7D).

The result indicated that the optimal read depth for both trio-based and duo-based analysis was 1-fold, regardless of read lengths (100 or 150 bp), sequencing modes (single-end or paired-end) and library construction methods (small-insert or mate-pair). For trio-based analysis, with the setting of 1-fold, paired-end sequencing at 100 bp and small-insert libraries, the average inconsistent rates of paternal inheritance among the five biological and five non-biological trios were 18.8% [standard deviation (SD): 1.89%] and 38.5% (SD: 1.19%), respectively, while the average inconsistent rates of maternal inheritance were 18.0% (SD: 3.03%) and 37.8% (SD: 1.12%), respectively (Table 1). In comparison, for duo-based mode with the same setting, the average inconsistent rate of paternal inheritance among the five biological and five non-biological trios were 18.5% (SD: 0.67%) and 38.4% (SD: 1.02%), respectively, while the average inconsistent rates of maternal inheritance were 18.3% (SD: 0.46%) and 37.9% (SD: 1.00%), respectively (Table 1). The inconsistent rate of paternal/maternal inheritance between two analytical modes was consistent. In comparison, in the setting of 1-fold, paired-end sequencing at 100 bp and mate-pair libraries, the results were highly consistent with the ones observed in the data from small-insert libraries. Therefore, the cutoff of reporting biological father/mother was 26.1% (Z>3) and 22.9% (Z>10) for trio-based and duo-based analysis, respectively.

To determine the turn-around-time (TAT) of LpPat when the data was with optimal setting (1-fold and paired-end 100 bp), the TAT required for each step was recorded. The total time required for the whole analysis was less than 1 hour (FIG. 3) for either mode for the analysis (trio-based or duo-based).

Validation of LpPat among 120 clinical trios and 50 trios from 1KGP was completed as follows. To validate LpPat's performance among different methods of library constructions and different sequencing platforms, the inventors randomly selected sequencing data from 170 trios, including 100 clinical trios sequenced with small-insert libraries from MGISeq-2000, 20 clinical trios sequenced with mate-pair libraries also from MGISeq-2000 and 50 trios sequenced with small-insert libraries from NovaSeq.

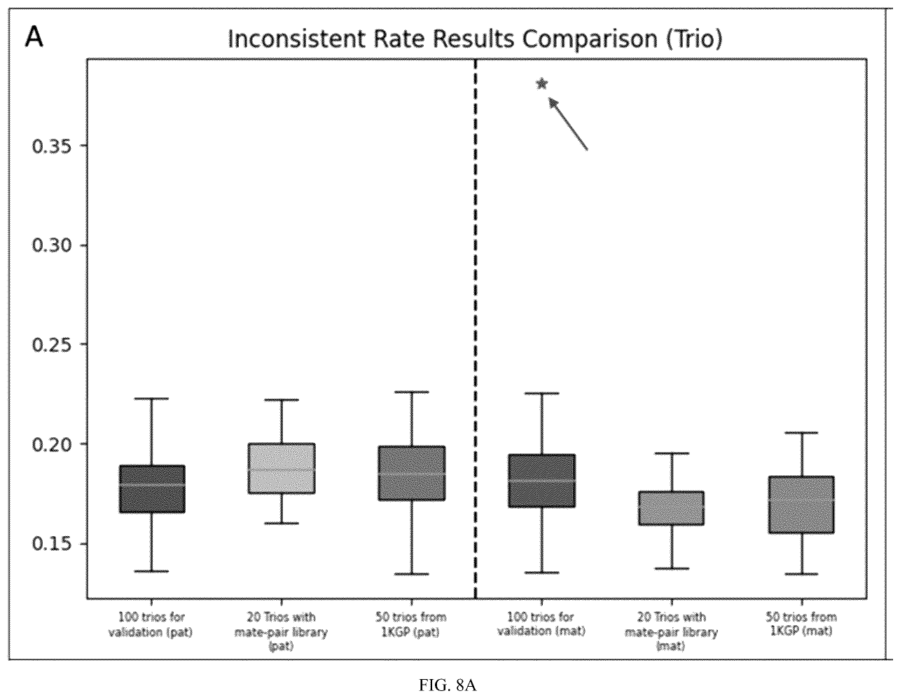

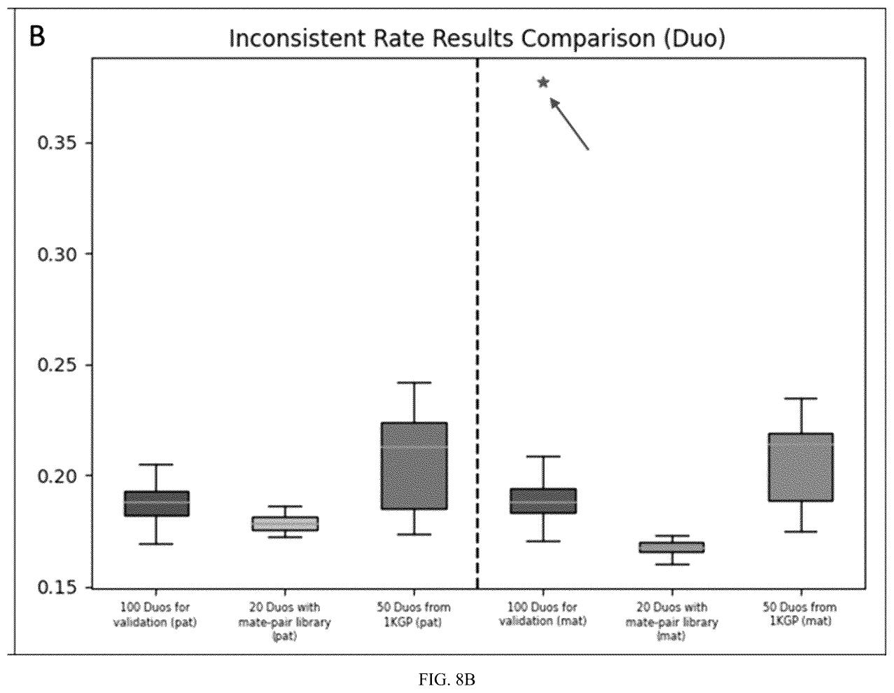

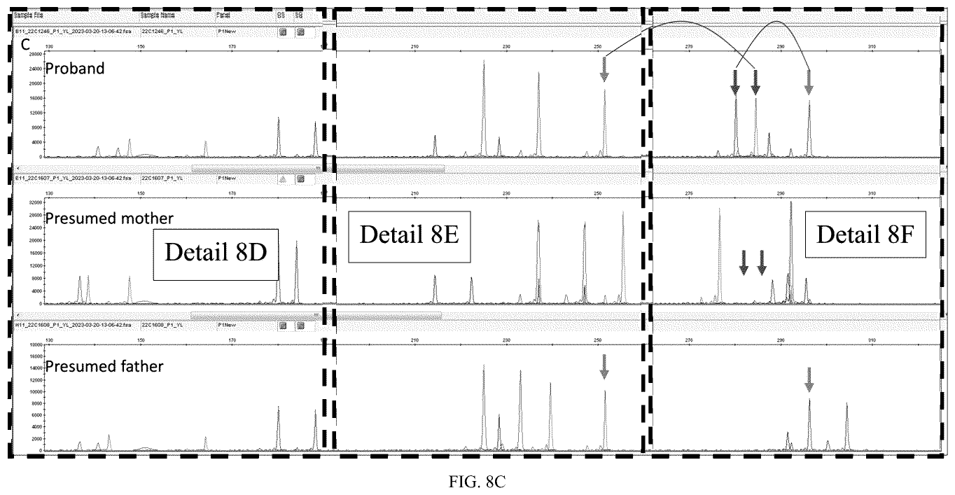

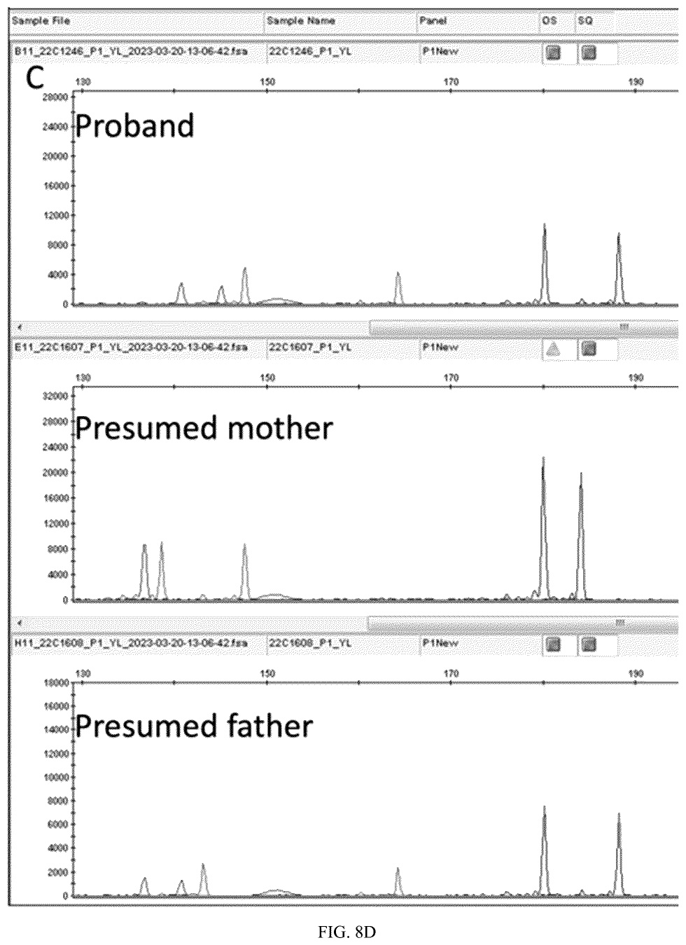

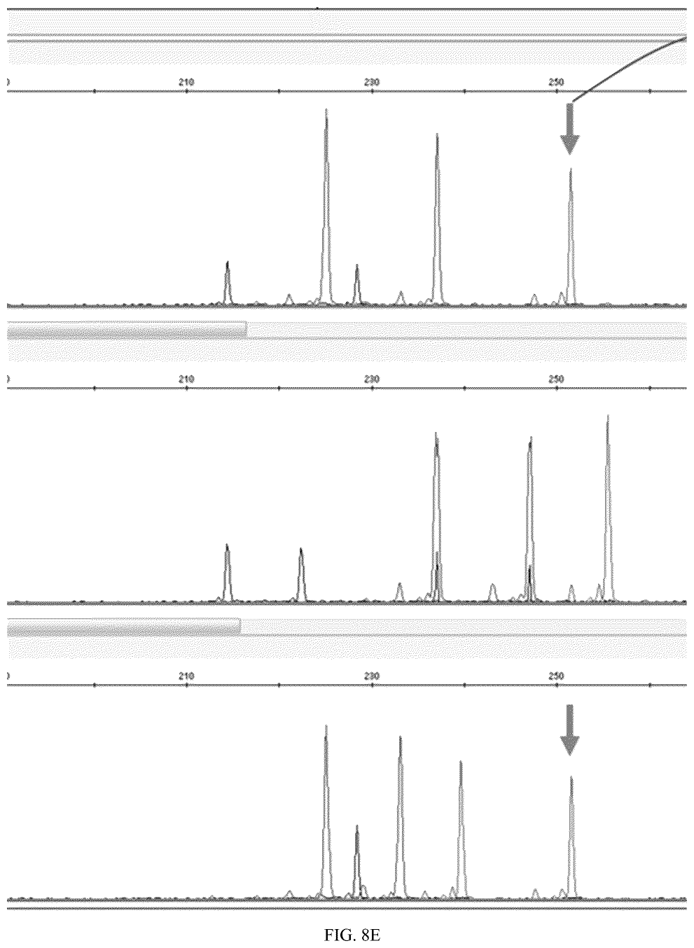

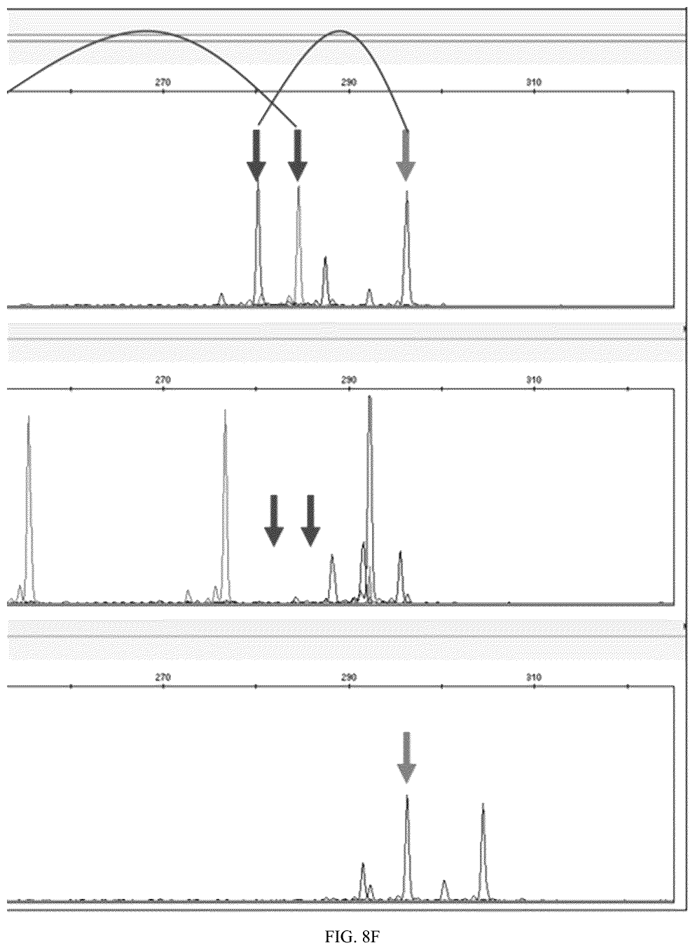

LpPat was performed in both trio and duo modes for determination of the paternal and maternal inheritance. Interestingly, all trios were reported as biological families except for case 22C1246. The inconsistent rates of maternal inheritance by trio-based and duo-based analysis were 38.1% and 37.7%, respectively, indicating the mother was not the non-biological mother. All clinical trios (n=120) were subjected for QF-PCR for paternity/maternity validation, while among the 50 trios from 1KGP, genotype information of those common SNPs among the proband and the presumed parents were used for the confirmation (FIGS. 8A-8F). For case 22C1246, STR marker confirmed that the mother was not the biological mother (FIGS. 8C-8F). The follow-up study indicated that the pregnancy was achieved by oocyte donation. The confirmation assays yielded a 100% consistent result with LpPat (FIGS. 8A-8B).

Investigation of recurrent SNVs likely resulted from systematic errors was completed as follows. As GS likely provides randomly distributed reads among the genome, those recurrent SNVs were likely resulted from systematic errors generated during alignment. It is contemplated within the scope of certain embodiments of the subject invention to investigate the presence of such recurrent SNVs with an optimal read-depth of 1-fold.

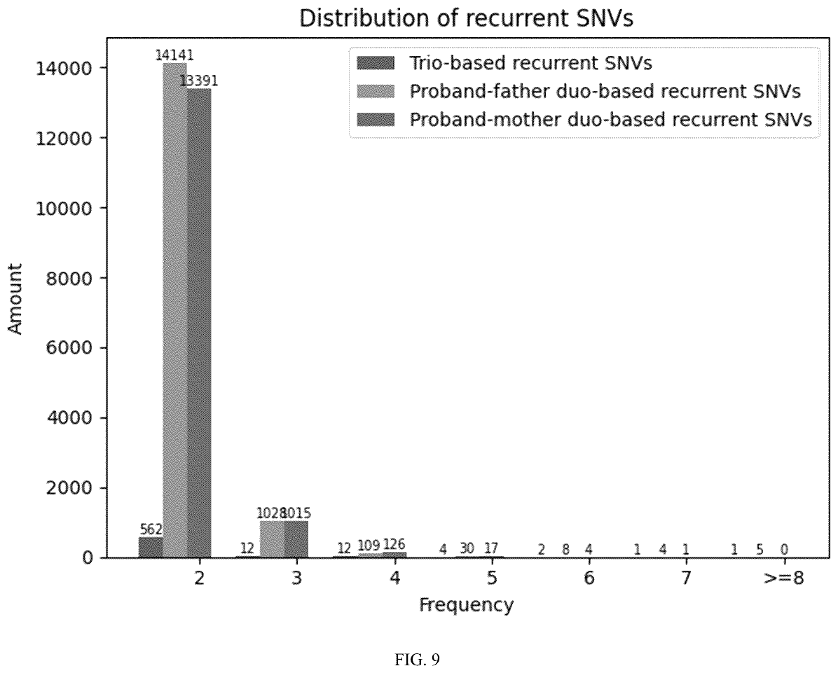

Among all 180 families, for trio-based analysis, the average number of loci that were homozygous in both parents but with different genotypes, and with 5 to 20 reads supporting in the proband was ˜707 for trio-based analysis. Among them, there were on average 126 SNVs were regarded as inconsistency of paternal/maternal inheritance in both paternity and maternity testing. Overall, 593 loci were detected more than once, among which only 70 loci occurred over twice (FIG. 9) and the average number of detecting recurrent SNVs per analysis was ˜5 (986/180) for paternity and ˜4 (763/180) for maternity. The percentage of detecting these recurrent SNVs per analysis was less than 1% (paternity: 5/707; maternity: 4/707).

For duo-based analysis, the average number of detected SNVs that were homozygous in the proband and the presumed father/mother was ˜11,158. In addition, an average of 2,097 SNVs were regarded as inconsistency of parental inheritance per analysis. 15,325 and 14,555 loci were detected more than once in proband-father and proband-mother analysis respectively (FIG. 9). Among them, up to 75% (11,523 for paternity and 13,391 for maternity) were detected twice. The percentage of these recurrent SNVs per test was ˜2.1% (229/11,158) in proband-father analysis and ˜2.0% (218/11,158) in proband-mother analysis.

This example features LpPat, a robust analytical pipeline based on low-pass GS for paternity testing according to certain embodiments of the subject invention. Embodiments provide a rapid (an overall TAT of <1 hour), platform neutral (regardless of sequencing parameters) and cost-effective (with read-depth of as low as 1-fold) paternity test, which can also serve as a QC step before subjecting for high read-depth GS analysis.

Low-pass GS has been widely used for germline structural variants detection (Raca, et al., 2023). However, it is limited in genotyping due to the insufficient coverage leading to the difficulty of paternity/maternity testing. Unlike STRs-based and SNP-based technologies, the accuracy of which were highly dependent on selection and amplification of specific genetic markers (Tam, et al., 2020; Zhang, et al., 2018) or the analysis being performed in trio-based or duo-based genome-wide mode. In addition, to minimize the effect of false positive or false negative detection of SNVs, embodiments established a baseline of inconsistent rate of paternal/maternal inheritance by using 10 trios with confirmed biological relationship and investigated the spectrum of inconsistent rate of paternal/maternal inheritance with non-paternity/maternity families by randomly assigning the parents to the probands. The robust performance was further confirmed by using 170 trios sequenced by different library constructions and sequencing platforms. To evaluate the effect contributed by systematic errors (such as alignment), the inventors identified 593 recurrent loci by trio-based analysis among all analyzed trios. There were only ˜1% of the overall available loci per test. In comparison, by duo-based analysis, due to the filter criteria of SNVs detection only required for two samples, nearly 10 times of loci were available for the analysis. However, the percentage of detecting recurrent SNVs was only ˜2% for paternity/maternity testing. Embodiments not only indicate that GS provided a randomly distributed coverage across the genome, but also demonstrated that the effect contributed by systematic errors was minimal. Embodiments established a database to include these recurrent loci and for further application, and those loci curated in this dataset would be filtered out.

Two modes were developed in certain embodiments: trio-based and duo-based, which were based on different hypotheses of variant inheritance. For each mode, the TAT was less than 1 hour. Although only one mode might be sufficient to indicate the paternity/maternity for each family, integration of two pipelines is also provided when there is a trio submitted in order to double confirmation of the results. In particular, two pipelines shared most of the analytical steps (such as alignment and reformatted); thus, the TAT of integration or running in parallel would be also less than 1 hour in certain embodiments.

It is noteworthy that families having children without genetic connections are more and more widespread due to the increasing rates of births involving gamete donation and surrogacy, together with adoptions (Casonato and Habersaat, 2015). According to ESHRE registries, more than 178,027 oocyte donation cycles had been performed only in Europe by 2011, and the number has steadily increased (Martinez, et al., 2021). Therefore, a quick and accurate paternity/maternity testing as QC test to confirm parentage for genetic diagnosis and to avoid sample mix-up is needed. Although this method serves as a QC step before subjecting to the high read-depth GS. In this example, all results have been confirmed, particularly for those 130 clinical trios that were confirmed by QF-PCR a gold-standard method. This indicates that embodiment is also able to provide a confirmation if the family only looks for a paternity/maternity test, although validation with a larger scale of sample size would be warranted.

Embodiments provide a rapid, cost-effective and platform neutral paternity/maternity test based on low-pass GS (as low as 1-fold read-depth) with two analytical modes provided (trio-based and duo-based), and demonstrate robust performance with data sequenced from different library construction methods and platform with further confirmation of the analytical results with QF-PCR.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

TABLE 1
Inconsistent rate of paternal or maternal inheritance
from GS data with 1-fold paired-end 100 bp in Phase I

| | Small-insert library | | Mate-pair library | |
|---|---|---|---|---|
| | Mean | SD | Mean | SD |
| | (%) | (%) | (%) | (%) |
| Biological trios (for paternity) | 18.7 | 1.89 | 18.5 | 1.33 |
| Biological trios (for maternity) | 18.0 | 3.03 | 17.2 | 1.05 |
| Trios with non-biological father | 38.5 | 1.19 | 44.9 | 1.76 |
| Trios with non-biological mother | 37.7 | 1.12 | 44.6 | 1.59 |
| Trios with neither of non- | 28.1 | 2.67 | 30.8 | 1.90 |
| biological parents | | | | |
| Biological duos: proband-father | 18.8 | 0.67 | 17.5 | 0.52 |
| Biological duos: proband-mother | 18.3 | 0.46 | 17.3 | 0.35 |
| Duos with non-biological father | 38.4 | 1.02 | 38.7 | 0.64 |
| Duos with non-biological mother | 37.9 | 1.00 | 38.1 | 0.79 |

*SD refers to standard deviation

TABLE 2
Evaluation of the optimal parameter with different sequencing parameters in trio-based analysis

| | | | | | | | | | Trios with | | Trios with | |
|---|---|---|---|---|---|---|---|---|---|---|---|---|
| | | | | | Trios with | | Trios with | | non-bio. | | non-bio. | |
| | Trios | | Trios | | non-bio. | | non-bio. | | fetus | | fetus | |
| | (paternity) | | (maternity) | | father | | mother | | (paternity) | | (maternity) | |
| | Mean | SD | Mean | SD | Mean | SD | Mean | SD | Mean | SD | Mean | SD |
| | (%) | (%) | (%) | (%) | (%) | (%) | (%) | (%) | (%) | (%) | (%) | (%) |
| 4X PE 150 bp | 12.7 | 0.40 | 12.7 | 0.51 | 51.9 | 3.97 | 49.9 | 0.45 | 34.9 | 4.49 | 30.9 | 2.69 |
| 3X PE 150 bp | 15.0 | 0.39 | 15.0 | 0.46 | 51.8 | 3.97 | 50.0 | 0.44 | 35.5 | 4.28 | 31.5 | 2.57 |
| 2X PE 150 bp | 17.3 | 0.40 | 17.0 | 0.5 | 50.9 | 3.85 | 49.3 | 0.63 | 35.5 | 4.12 | 32.2 | 2.92 |
| 1X PE 150 bp | 17.3 | 2.31 | 16.3 | 2.45 | 35.5 | 5.64 | 36.1 | 4.10 | 27.4 | 3.40 | 27.7 | 3.29 |
| 4X PE 100 bp | 12.2 | 0.32 | 12.3 | 0.28 | 50.1 | 0.74 | 49.9 | 0.35 | 32.7 | 0.69 | 32.3 | 0.69 |
| 3X PE 100 bp | 14.7 | 0.35 | 14.7 | 0.31 | 50.1 | 0.56 | 50.0 | 0.25 | 33.5 | 0.59 | 32.9 | 0.60 |
| 2X PE 100 bp | 17.3 | 0.56 | 17.0 | 0.16 | 49.8 | 0.89 | 49.4 | 0.41 | 33.8 | 0.65 | 33.2 | 0.70 |
| 1X PE 100 bp | 18.8 | 1.89 | 18.0 | 3.03 | 38.47 | 1.19 | 37.7 | 1.12 | 23.5 | 2.61 | 28.8 | 2.57 |
| 0.5XPE100 bp | 11.8 | 4.06 | 12.3 | 3.51 | 17.1 | 3.01 | 12.2 | 4.86 | 13.7 | 4.22 | 13.1 | 4.26 |
| 4X SE 150 bp | 12.1 | 0.38 | 11.9 | 0.32 | 52.1 | 3.93 | 50.0 | 0.31 | 35.1 | 4.50 | 30.7 | 2.84 |
| 3X SE 150 bp | 14.6 | 0.46 | 14.4 | 0.38 | 52.2 | 3.94 | 50.0 | 0.31 | 35.8 | 4.44 | 31.4 | 2.84 |
| 2X SE 150 bp | 17.1 | 0.85 | 16.8 | 0.52 | 51.7 | 38.4 | 49.6 | 0.71 | 35.9 | 4.26 | 31.9 | 2.67 |
| 1X SE 150 bp | 17.8 | 0.79 | 16.4 | 3.26 | 40.1 | 3.08 | 35.3 | 3.14 | 30.0 | 4.73 | 26.4 | 3.65 |
| 1X PE 100 bp | 18.5 | 1.33 | 17.2 | 1.05 | 44.9 | 1.76 | 44.6 | 1.59 | 30.5 | 1.88 | 31.1 | 1.88 |
| (mate-pair | | | | | | | | | | | | |
| library) | | | | | | | | | | | | |

*SD refers to standard deviation; PE refers to paired-end, SE refers to single-end; X refers to read fold.

TABLE 3
Evaluation of the optimal parameter with different
sequencing parameters in duo-based analysis

| | | | | | Duos | | Duos | |
|---|---|---|---|---|---|---|---|---|
| | Duos | | Duos | | with non- | | with non- | |
| | (for | | (for | | biological | | biological | |
| | paternity) | | maternity) | | father | | mother | |
| | Mean | SD | Mean | SD | Mean | SD | Mean | SD |
| | (%) | (%) | (%) | (%) | (%) | (%) | (%) | (%) |
| 4X PE 150 bp | 6.3 | 0.22 | 6.4 | 0.24 | 32.3 | 0.44 | 32.1 | 0.25 |
| 3X PE 150 bp | 9.8 | 1.24 | 9.2 | 0.38 | 34.8 | 0.80 | 34.2 | 0.26 |
| 2X PE 150 bp | 12.7 | 0.36 | 12.6 | 0.43 | 37.0 | 0.56 | 36.6 | 0.35 |
| 1X PE 150 bp | 16.7 | 0.36 | 16.5 | 0.66 | 38.8 | 0.37 | 38.6 | 0.63 |
| 4X PE 100 bp | 9.9 | 0.31 | 10.9 | 0.20 | 35.3 | 0.60 | 35.0 | 0.25 |
| 3X PE 100 bp | 12.5 | 0.4 | 12.4 | 0.32 | 37.3 | 0.64 | 37.0 | 0.27 |
| 2X PE 100 bp | 15.7 | 0.59 | 39.5 | 0.81 | 15.4 | 0.33 | 39.0 | 0.39 |
| 1X PE 100 bp | 18.8 | 0.67 | 18.3 | 0.46 | 38.4 | 1.02 | 37.9 | 1.00 |
| 0.5XPE100 bp | 15.1 | 1.41 | 13.4 | 2.04 | 22.1 | 2.11 | 21.1 | 0.71 |
| 4X SE 150 bp | 12.3 | 0.46 | 12.0 | 0.33 | 36.8 | 0.81 | 36.4 | 0.30 |
| 3X SE 150 bp | 14.5 | 0.67 | 14.2 | 0.39 | 38.4 | 0.99 | 37.8 | 0.32 |
| 2X SE 150 bp | 15.7 | 0.59 | 15.4 | 0.33 | 39.5 | 0.81 | 39.0 | 0.39 |
| 1X SE 150 bp | 18.7 | 2.10 | 17.3 | 0.55 | 34.7 | 2.73 | 32.4 | 1.17 |
| 1X PE 100 bp | 17.5 | 0.52 | 17.3 | 0.35 | 38.7 | 0.64 | 38.1 | 0.79 |
| (mate-pair | | | | | | | | |
| library) | | | | | | | | |

*SD refers to standard deviation; PE refers to paired-end, SE refers to single-end; X refers to read fold.

### Exemplary Embodiments

Embodiment 1. A method to determine paternity, maternity, or parentage of a subject, the method comprising:
(i) aligning sequence reads from low-pass genome sequencing data equivalent to 1-fold or more read-depth of genomic DNA of biological samples to a human genome reference according to an aligned chromosome and one or more genomic coordinates yielding a respective aligned sequence read;
(ii) identifying a multiplicity of single-nucleotide variants (SNVs) in each respective aligned sequence read, wherein an SNV at each site has a mutant base type different from a base type at a corresponding site from a human genome reference;
(iii) identifying a number of homozygous SNVs and a number of diploid heterozygous SNVs from the multiplicity of SNVs identified in step (ii), wherein a homozygous SNV is identified as an SNV where a percentage of sequence reads supporting the mutant base type different from the base type at the corresponding site from the human genome reference is 100%, and a diploid heterozygous SNV is identified as an SNV where the percentage of sequence reads supporting the mutant base type different from the base type at the corresponding site from the human genome reference is at least about 25% and less than about 75%; and
(iv) determining an inconsistent rate of base-type inheritance from a number of homozygous SNVs and a number of diploid heterozygous SNVs identified in step (iii) for two analytical models comprising a trio-based analysis model comprising a proband and two parents, a mother and a father; and a duo-based analysis model comprising a proband and one parent, a mother or a father, wherein:
   for the trio-based analysis, (a) a number of loci that both the mother and the father are in homozygous alignment but with different genotypes in the i-^{th} chromosome is denoted as Aⁱ; (b) among the members denoted in (a), the number of SNVs that are homozygous in the proband but with different genotypes from the presumed father is denoted as pi for a paternity test and the number of SNVs that are homozygous in the proband but with different genotypes from the presumed mother is denoted as mi: for a maternity test; (c) the inconsistent rate of paternal inheritance αi in chromosome i is calculated based on formula (1), and the inconsistent rate of maternal inheritance βi in chromosome i is calculated based on formula (2);

$$α i = \frac{pi}{Ai} \qquad ( 1 )$$

$$β i = \frac{mi}{Ai} \qquad ( 2 )$$

(d) the paternity is determined by using an average rate λ̅pat across all autosomal chromosomes based on formula (3), and the maternity is determined by using an average rate λ̅mat across all autosomal chromosomes based on formula (4);

$$\overline{λ} pat = \frac{{\sum }_{i = 1}^{n = 22} α i}{22} \qquad ( 3 )$$

$$\overline{λ} mat = \frac{{\sum }_{i = 1}^{n = 22} β i}{22} \qquad ( 4 )$$

for duo-based analysis, (a) the number of homozygous SNVs in both proband and the presumed parent in chromosome i is denoted Adi; (b) among the members denoted in (a), the number of homozygous SNVs that are with different genotypes between the proband and the presumed parent is denoted as qi; (c) the inconsistent rate γi of parental inheritance in chromosome i is calculated based on formula (5);

$$γ i = \frac{qi}{Adi} \qquad ( 5 )$$

(d) the respective paternity or maternity is determined using an average rate parent across all autosomal chromosomes based on formula (6);

$$\overline{λ} parent = \frac{{\sum }_{i = 1}^{n = 22} γ i}{22} \qquad ( 6 )$$

for determining the paternity, maternity, or parentage.

Embodiment 2. The method of embodiment 1, wherein the biological sample is selected from the group consisting of peripheral blood, chorionic villus, amniotic fluid, cord blood, placental tissue, and tissue samples from organs.

Embodiment 3. The method of any preceding embodiment, wherein the subject is a pregnant female, a non-pregnant female, an infant, or a male with a need to confirm paternity or maternity.

Embodiment 4. The method of any preceding embodiment, wherein the multiplicity of sequence reads comprise single-end sequence reads or paired-end sequence reads.

Embodiment 5. The method of any preceding embodiment, wherein the low-pass genome sequencing has a read depth of 1 fold to 15 folds.

Embodiment 6. The method of any preceding embodiment, wherein the human genome reference is GRCh37/hg19, GRCh38/hg38, or T2T-CHM13v2.0.

Embodiment 7. The method of any preceding embodiment, wherein the aligning step is performed using Short Oligonucleotide Alignment Program 2 (SOAP2) or Burrows-Wheeler Aligner (BWA) and Bowtie2.

Embodiment 8. The method of any preceding embodiment, wherein step (ii) further comprises removing one or more sequence reads generated by polymerase chain reaction (PCR) duplication.

Embodiment 9. The method of any preceding embodiment, wherein step (iii) further comprises discarding a site selected from the group consisting of:
(a) a site wherein a minimal read-depth of the site is determined by the minimal read-depth of the biological sample;
(b) a site wherein a maximum read-depth of the site is determined by the maximal read-depth of the biological sample; and
(c) a site where no sequence read supports a mutant base type.

Embodiment 10. The method of any preceding embodiment, wherein step (iv) comprises determining the paternity or maternity determination by comparing the inconsistent rate with a cutoff value determined by a process comprising a comparison of a biological-inconsistent rate of parental inheritance among a group of biological families against a non-biological-inconsistent rate of parental inheritance among a group of simulated non-paternity/non-maternity families.

Embodiment 11. A computer system for determination of paternity or maternity in a trio of a subject, comprising a processor operably connected to a memory storing a plurality of instructions, wherein the processor, upon processing the instructions, performs the following steps:
(i) aligning at least two sequence reads from low-pass genome sequencing of genomic DNA of biological samples to a human genome reference according to an aligned chromosome and one or more genomic coordinates yielding a respective aligned sequence read;
(ii) identifying a multiplicity of single-nucleotide variants (SNVs) in each respective aligned sequence read, wherein an SNV at each site has a mutant base type different from a base type at the corresponding site from the human genome reference;
(iii) identifying a number of homozygous SNVs and a number of diploid heterozygous SNVs from the multiplicity of SNVs identified in step (ii), wherein
   a homozygous SNV is identified as an SNV where a percentage of sequence reads supporting the mutant base type different from a base type at a corresponding site from the human genome reference is 100%, and
   a diploid heterozygous SNV is identified as an SNV where a percentage of sequence reads supporting a mutant base type different from a base type at a corresponding site from the human genome reference is at least about 25% and no larger than 75%; and
(iv) determining an inconsistent rate of base-type inheritance from the number of homozygous SNVs and the number of diploid heterozygous SNVs identified in step (iii) for an analytical model comprising a trio-based analysis comprising a proband and two parents, a mother and a father, wherein:
for the trio-based analysis, (a) a number of loci that both the mother and the father are in homozygous alignment but with different genotypes in the i-^{th} chromosome is denoted as Aⁱ; (b) among the members denoted in (a), the number of SNVs that are homozygous in the proband but with different genotypes from the presumed father is denoted as pi for a paternity test and the number of SNVs that are homozygous in the proband but with different genotypes from the presumed mother is denoted as mi: for a maternity test; (c) the inconsistent rate of paternal inheritance αi in chromosome i is calculated based on the formula (1), and the inconsistent rate of maternal inheritance βi in chromosome i is calculated based on the formula (2);

$$α i = \frac{pi}{Ai} \qquad ( 1 )$$

$$β i = \frac{mi}{Ai} \qquad ( 2 )$$

(d) the paternity is determined by using an average rate λ̅pat across all autosomal chromosomes based on formula (3), and the maternity is determined by using an average rate λ̅mat across all autosomal chromosomes based on formula (4);

$$\overline{λ} pat = \frac{{\sum }_{i = 1}^{n = 22} α i}{22} \qquad ( 3 )$$

$$\overline{λ} mat = \frac{{\sum }_{i = 1}^{n = 22} β i}{22} \qquad ( 4 )$$

for providing the determination of paternity or maternity in a trio.

Embodiment 12. The computer system of embodiment 11, wherein the biological sample is selected from the group consisting of peripheral blood, chorionic villus, amniotic fluid, cord blood, placental tissue, and tissue samples from organs.

Embodiment 13. The computer system of any preceding embodiment, wherein the subject is a pregnant female, a non-pregnant female, an infant, or a male having a need to confirm paternity or maternity.

Embodiment 14. The computer system of any preceding embodiment, wherein the multiplicity of sequence reads comprise single-end sequence reads, paired-end sequence reads, or both.

Embodiment 15. The computer system of any preceding embodiment, wherein the low-pass genome sequencing has a read depth in a range of from 1 fold to 15 folds.

Embodiment 16. The computer system of any preceding embodiment, wherein the human genome reference is GRCh37/hg19, GRCh38/hg38, or T2T-CHM13v2.0.

Embodiment 17. The computer system of any preceding embodiment, wherein the aligning operation comprises application of Short Oligonucleotide Alignment Program 2 (SOAP2); or application of Burrows-Wheeler Aligner (BWA) and Bowtie2.

Embodiment 18. The computer system of any preceding embodiment, wherein the processor, upon processing the instructions, is further configured to remove sequence reads generated by polymerase chain reaction (PCR) duplication.

Embodiment 19. The computer system of any preceding embodiment, wherein the processor, upon processing the instructions, is further configured to discard a site exhibiting at least one property selected from the group comprising:
(a) a minimal read-depth of the site that is equal to a minimal read-depth of the biological sample;
(b) a maximum read-depth of the site that is equal to a maximal read-depth of the biological sample; and
(c) no sequence read at the site that supports a mutant base type.

Embodiment 20. The computer system of any preceding embodiment, wherein the processor, upon processing the instructions, is further configured to identify paternity or maternity by comparing an inconsistent rate calculation with a predetermined cutoff.

### REFERENCES

Byrska-Bishop, M., et al. High-coverage whole-genome sequencing of the expanded 1000 Genomes Project cohort including 602 trios. Cell 2022; 185(18):3426-3440 e3419.
Cao, Y., et al. Exploring the diagnostic utility of genome sequencing for fetal congenital heart defects. Prenat Diagn 2022; 42(7):862-872.
Casonato, M. and Habersaat, S. Parenting without being genetically connected. Enfance 2015; 3(3):289-306.
Chandra, D., et al. Mutation rate evaluation at 21 autosomal STR loci: Paternity testing experience. Leg Med (Tokyo) 2022; 58:102080.
Chaubey, A., et al. Low-Pass Genome Sequencing: Validation and Diagnostic Utility from 409 Clinical Cases of Low-Pass Genome Sequencing for the Detection of Copy Number Variants to Replace Constitutional Microarray. J Mol Diagn 2020; 22(6):823-840.
Choy, K. W., et al. Prenatal Diagnosis of Fetuses With Increased Nuchal Translucency by Genome Sequencing Analysis. Front Genet 2019; 10:761.
Dong, Z., et al. Deciphering the complexity of simple chromosomal insertions by genome sequencing. Hum Genet 2021; 140(2):361-380.
Dong, Z., et al. Low-pass genome sequencing-based detection of absence of heterozygosity: validation in clinical cytogenetics. Genet Med 2021; 23(7):1225-1233.
Dong, Z., et al. Mate-pair genome sequencing reveals structural variants for idiopathic male infertility. Hum Genet 2023; 142(3):363-377.
Dong, Z., et al. Identification of balanced chromosomal rearrangements previously unknown among participants in the 1000 Genomes Project: implications for interpretation of structural variation in genomes and the future of clinical cytogenetics. Genet Med 2018; 20(7):697-707.
Dong, Z., et al. Low-pass whole-genome sequencing in clinical cytogenetics: a validated approach. Genet Med 2016; 18(9):940-948.
Dong, Z., et al. Development of coupling controlled polymerizations by adapter-ligation in mate-pair sequencing for detection of various genomic variants in one single assay. DNA Res 2019; 26(4):313-325.
Li, H. and Durbin, R. Fast and accurate short read alignment with Burrows-Wheeler transform. Bioinformatics 2009; 25(14):1754-1760.
Li, H., et al. The Sequence Alignment/Map format and SAMtools. Bioinformatics 2009; 25(16):2078-2079.
Liang, D., et al. Copy number variation sequencing for comprehensive diagnosis of chromosome disease syndromes. J Mol Diagn 2014; 16(5):519-526.
Martinez, F., et al. Ovarian stimulation for oocyte donation: a systematic review and meta-analysis. Hum Reprod Update 2021; 27(4):673-696.
Ou, X. and Qu, N. Noninvasive prenatal paternity testing by target sequencing microhaps. Forensic Sci Int Genet 2020; 48:102338.
Prero, M. Y., et al. Disclosure of Misattributed Paternity. Pediatrics 2019; 143(6).
Prokop, J. W., et al. Genome sequencing in the clinic: the past, present, and future of genomic medicine. Physiol Genomics 2018; 50(8):563-579.
Raca, G., et al. Points to consider in the detection of germline structural variants using next-generation sequencing: A statement of the American College of Medical Genetics and Genomics (ACMG). Genet Med 2023; 25(2):100316.
Redin, C., et al. The genomic landscape of balanced cytogenetic abnormalities associated with human congenital anomalies. Nat Genet 2017; 49(1):36-45.
Schwark, T., et al. The SNPforID Assay as a Supplementary Method in Kinship and Trace Analysis. Transfus Med Hemother 2012; 39(3):187-193.
Shen, X., et al. Noninvasive Prenatal Paternity Testing with a Combination of Well-Established SNP and STR Markers Using Massively Parallel Sequencing. Genes (Basel) 2021; 12(3).
Stefka, J., et al. Misattributed parentage identified through diagnostic exome sequencing: Frequency of detection and reporting practices. J Genet Couns 2022; 31(3):631-640.
Tam, J. C. W., et al. Noninvasive prenatal paternity testing by means of SNP-based targeted sequencing. Prenat Diagn 2020; 40(4):497-506.
Wang, H., et al. Low-pass genome sequencing versus chromosomal microarray analysis: implementation in prenatal diagnosis. Genet Med 2020; 22(3):500-510.
Zhang, S., et al. Non-invasive prenatal paternity testing using cell-free fetal DNA from maternal plasma: DNA isolation and genetic marker studies. Leg Med (Tokyo) 2018; 32:98-103.
Zhou, J., et al. Whole Genome Sequencing in the Evaluation of Fetal Structural Anomalies: A Parallel Test with Chromosomal Microarray Plus Whole Exome Sequencing. Genes (Basel) 2021; 12(3).

We claim:

1. A method to determine paternity, maternity, or parentage of a subject, the method comprising:
(i) aligning sequence reads from low-pass genome sequencing data equivalent to 1-fold or more read-depth of genomic DNA of biological samples to a human genome reference according to an aligned chromosome and one or more genomic coordinates yielding a respective aligned sequence read;
(ii) identifying a multiplicity of single-nucleotide variants (SNVs) in each respective aligned sequence read, wherein an SNV at each site has a mutant base type different from a base type at a corresponding site from a human genome reference;
(iii) identifying a number of homozygous SNVs and a number of diploid heterozygous SNVs from the multiplicity of SNVs identified in step (ii), wherein a homozygous SNV is identified as an SNV where a percentage of sequence reads supporting the mutant base type different from the base type at the corresponding site from the human genome reference is 100%, and a diploid heterozygous SNV is identified as an SNV where the percentage of sequence reads supporting the mutant base type different from the base type at the corresponding site from the human genome reference is at least about 25% and less than about 75%; and
(iv) determining an inconsistent rate of base-type inheritance from a number of homozygous SNVs and a number of diploid heterozygous SNVs identified in step (iii) for two analytical models comprising a trio-based analysis model comprising a proband and two parents, a mother and a father; and a duo-based analysis model comprising a proband and one parent, a mother or a father, wherein:
for the trio-based analysis, (a) a number of loci that both the mother and the father are in homozygous alignment but with different genotypes in the i-^{th} chromosome is denoted as Aⁱ; (b) among the members denoted in (a), the number of SNVs that are homozygous in the proband but with different genotypes from the presumed father is denoted as pi for a paternity test and the number of SNVs that are homozygous in the proband but with different genotypes from the presumed mother is denoted as mi: for a maternity test; (c) the inconsistent rate of paternal inheritance αi in chromosome i is calculated based on formula (1), and the inconsistent rate of maternal inheritance βi in chromosome i is calculated based on formula (2);
$$α i = \frac{pi}{Ai} \qquad ( 1 )$$
$$β i = \frac{mi}{Ai} \qquad ( 2 )$$
(d) the paternity is determined by using an average rate λ̅pat across all autosomal chromosomes based on formula (3), and the maternity is determined by using an average rate λ̅mat across all autosomal chromosomes based on formula (4);
$$\overline{λ} pat = \frac{{\sum }_{i = 1}^{n = 22} α i}{22} \qquad ( 3 )$$
$$\overline{λ} mat = \frac{{\sum }_{i = 1}^{n = 22} β i}{22} \qquad ( 4 )$$
for duo-based analysis, (a) the number of homozygous SNVs in both proband and the presumed parent in chromosome i is denoted Adi; (b) among the members denoted in (a), the number of homozygous SNVs that are with different genotypes between the proband and the presumed parent is denoted as qi; (c) the inconsistent rate γi of parental inheritance in chromosome i is calculated based on formula (5);
$$γ i = \frac{qi}{Adi} \qquad ( 5 )$$
(d) the respective paternity or maternity is determined using an average rate λ̅parent across all autosomal chromosomes based on formula (6);
$$\overline{λ} parent = \frac{{\sum }_{i = 1}^{n = 22} γ i}{22} \qquad ( 6 )$$
for determining the paternity, maternity, or parentage.

2. The method of claim 1, wherein the biological sample is selected from the group consisting of peripheral blood, chorionic villus, amniotic fluid, cord blood, placental tissue, and tissue samples from organs.

3. The method of claim 1, wherein the subject is a pregnant female, a non-pregnant female, an infant, or a male with a need to confirm paternity or maternity.

4. The method of claim 1, wherein the multiplicity of sequence reads comprise single-end sequence reads or paired-end sequence reads.

5. The method of claim 1, wherein the low-pass genome sequencing has a read depth of 1 fold to 15 folds.

6. The method of claim 5, wherein the human genome reference is GRCh37/hg19, GRCh38/hg38, or T2T-CHM13v2.0.

7. The method of claim 5, wherein the aligning step is performed using Short Oligonucleotide Alignment Program 2 (SOAP2) or Burrows-Wheeler Aligner (BWA) and Bowtie2.

8. The method of claim 1, wherein step (ii) further comprises removing one or more sequence reads generated by polymerase chain reaction (PCR) duplication.

9. The method of claim 1, wherein step (iii) further comprises discarding a site selected from the group consisting of:
(a) a site wherein a minimal read-depth of the site is determined by the minimal read-depth of the biological sample;
(b) a site wherein a maximum read-depth of the site is determined by the maximal read-depth of the biological sample; and
(c) a site where no sequence read supports a mutant base type.

10. The method of claim 1, wherein step (iv) comprises determining the paternity or maternity determination by comparing the inconsistent rate with a cutoff value determined by a process comprising a comparison of a biological-inconsistent rate of parental inheritance among a group of biological families against a non-biological-inconsistent rate of parental inheritance among a group of simulated non-paternity/non-maternity families.

11. A computer system for determination of paternity or maternity in a trio of a subject, comprising a processor operably connected to a memory storing a plurality of instructions, wherein the processor, upon processing the instructions, performs the following steps:
(i) aligning at least two sequence reads from low-pass genome sequencing of genomic DNA of biological samples to a human genome reference according to an aligned chromosome and one or more genomic coordinates yielding a respective aligned sequence read;
(ii) identifying a multiplicity of single-nucleotide variants (SNVs) in each respective aligned sequence read, wherein an SNV at each site has a mutant base type different from a base type at the corresponding site from the human genome reference;
(iii) identifying a number of homozygous SNVs and a number of diploid heterozygous SNVs from the multiplicity of SNVs identified in step (ii), wherein
a homozygous SNV is identified as an SNV where a percentage of sequence reads supporting the mutant base type different from a base type at a corresponding site from the human genome reference is 100%, and
a diploid heterozygous SNV is identified as an SNV where a percentage of sequence reads supporting a mutant base type different from a base type at a corresponding site from the human genome reference is at least about 25% and no larger than 75%; and
(iv) determining an inconsistent rate of base-type inheritance from the number of homozygous SNVs and the number of diploid heterozygous SNVs identified in step (iii) for an analytical model comprising a trio-based analysis comprising a proband and two parents, a mother and a father, wherein:
for the trio-based analysis, (a) a number of loci that both the mother and the father are in homozygous alignment but with different genotypes in the i-^{th} chromosome is denoted as Aⁱ; (b) among the members denoted in (a), the number of SNVs that are homozygous in the proband but with different genotypes from the presumed father is denoted as pi for a paternity test and the number of SNVs that are homozygous in the proband but with different genotypes from the presumed mother is denoted as mi: for a maternity test; (c) the inconsistent rate of paternal inheritance αi in chromosome i is calculated based on the formula (1), and the inconsistent rate of maternal inheritance βi in chromosome i is calculated based on the formula (2);
$$α i = \frac{pi}{Ai} \qquad ( 1 )$$
$$β i = \frac{mi}{Ai} \qquad ( 2 )$$
(d) the paternity is determined by using an average rate λ̅pat across all autosomal chromosomes based on formula (3), and the maternity is determined by using an average rate λ̅mat across all autosomal chromosomes based on formula (4);
$$\overline{λ} pat = \frac{{\sum }_{i = 1}^{n = 22} α i}{22} \qquad ( 3 )$$
$$\overline{λ} mat = \frac{{\sum }_{i = 1}^{n = 22} β i}{22} \qquad ( 4 )$$
for providing the determination of paternity or maternity in a trio.

12. The computer system of claim 11, wherein the biological sample is selected from the group consisting of peripheral blood, chorionic villus, amniotic fluid, cord blood, placental tissue, and tissue samples from organs.

13. The computer system of claim 11, wherein the subject is a pregnant female, a non-pregnant female, an infant, or a male having a need to confirm paternity or maternity.

14. The computer system of claim 13, wherein the multiplicity of sequence reads comprise single-end sequence reads, paired-end sequence reads, or both.

15. The computer system of claim 14, wherein the low-pass genome sequencing has a read depth in a range of from 1 fold to 15 folds.

16. The computer system of claim 15, wherein the human genome reference is GRCh37/hg19, GRCh38/hg38, or T2T-CHM13v2.0.

17. The computer system of claim 11, wherein the aligning operation comprises application of Short Oligonucleotide Alignment Program 2 (SOAP2); or application of Burrows-Wheeler Aligner (BWA) and Bowtie2.

18. The computer system of claim 16, wherein the processor, upon processing the instructions, is further configured to remove sequence reads generated by polymerase chain reaction (PCR) duplication.

19. The computer system of claim 18, wherein the processor, upon processing the instructions, is further configured to discard a site exhibiting at least one property selected from the group comprising:
(a) a minimal read-depth of the site that is equal to a minimal read-depth of the biological sample;
(b) a maximum read-depth of the site that is equal to a maximal read-depth of the biological sample; and
(c) no sequence read at the site that supports a mutant base type.

20. The computer system of claim 10, wherein the processor, upon processing the instructions, is further configured to identify paternity or maternity by comparing an inconsistent rate calculation with a predetermined cutoff.
